(19) United States
(12) Patent Application Publication
(10) Pub. No.: US 2026/0062713 A1
(43) Pub. Date: Mar. 5, 2026

(54) ADENO-ASSOCIATED VIRUS DELIVERY OF CLN3 POLYNUCLEOTIDE

(71) Applicants: RESEARCH INSTITUTE AT NATIONWIDE CHILDREN'S HOSPITAL, Columbus, OH (US); OHIO STATE INNOVATION FOUNDATION, Columbus, OH (US)

(72) Inventors: Kathrin Meyer, Columbus, OH (US); Brian K. Kaspar, Westerville, OH (US); Kevin Foust, Columbus, OH (US)

(21) Appl. No.: 19/289,974

(22) Filed: Aug. 4, 2025

Related U.S. Application Data
(62) Division of application No. 17/427,010, now Pat. No. 12,404,523.
(60) Provisional application No. 62/800,911, filed on Feb. 4, 2019.

(51) Int. Cl. C12N 15/86; A61P 3/00; A61P 25/00; C07K 14/47; C12N 7/00

(52) U.S. Cl. CPC C12N 15/86; A61P 3/00; A61P 25/00; C07K 14/47; C12N 7/00; C12N 2750/14143; C12N 2750/14171

(57) ABSTRACT

The present disclosure relates to recombinant adeno-associated virus (rAAV) delivery of a ceroid lipofuscinosis neuronal 3 (CLN3) polynucleotide. The disclosure provides rAAV and methods of using the rAAV for CLN3 gene therapy of the neuronal ceroid lipofuscinosis or CLN3-Batten Discase.

This application is a divisional of U.S. patent application Ser. No. 17/427,010 filed Jul. 29, 2021, which is a U.S. national phase application of International Application No. PCT/US20/16542 which claims priority to U.S. Provisional Patent Application No. 62/800,911, filed Feb. 4, 2019, all of which are incorporated by reference herein in their entirety.

### INCORPORATION BY REFERENCE OF THE SEQUENCE LISTING

This application contains, as a separate part of disclosure, a Sequence Listing in computer-readable form (filename: 53576A_SeqListing.txt; 31,123 bytes, created Jul. 25, 2025) which is incorporated by reference herein in its entirety.

### FIELD

The present disclosure relates to recombinant adeno-associated virus (rAAV) delivery of a ceroid lipofuscinosis neuronal 3 (CLN3) polynucleotide. The disclosure provides rAAV and methods of using the rAAV for CLN3 gene therapy of the neuronal ceroid lipofuscinosis (NCL) or CLN3-Batten Disease.

### BACKGROUND

Neuronal ceroid lipofuscinoses (NCLs) are a group of severe neurodegenerative disorders.

Mutations in the CLN3 gene cause juvenile NCL or CLN3-Batten Disease (Kitzmü et al., Human Molecular Genetics 2008; 17(2):303-312; Munroe et al., Am J Hum Genet. 1997; 61:310-316), which has also been called Spielmeyer-Sjogren-Vogt disease. Mutations disturb the lysosomal storage clearance process. At this time, 67 disease causing-mutations have been described. However, 85% of patients are homozygous for the 1.02 kb deletion leading to the loss of exon 7 and exon 8. CLN3 mutations found in patients predominantly cause reduced abundance or functionality of the protein (battenin).

The typical age of onset in CLN3-Batten disease is between 4-7 years with insidious, but rapidly progressive vision loss. Children with juvenile NCL go from having normal vision to blindness in a matter of months, but can maintain light-dark perception for several years after. Cognitive and motor decline usually follows next (7-10 years of age) alongside with behavioral problems such as aggression (8-10 years of age), and then seizures (10-12 years of age). Parkinsonian features develop between 11-13 years of age. Cardiac conduction abnormalities have been reported in individuals at later stages of the disease. There is high phenotypic variability in individuals affected with CLN3-Batten disease, but all have low vision or progressive blindness in common. Moreover, the physical subscale of the Unified Batten Disease Rating Scale (UBDRS) that has been validated in 82 patients, shows a steady and measurable decline of 2.86 points per year (2.27-3.45, p<0.0001). The average survival is usually 15 years from symptom onset to end of life.

Therapeutic measures for CLN3-Batten disease have been wide-ranging in an effort to ameliorate disease. These include drug therapy such as EGIS-8332 and talampanel which target AMPA receptors, drugs that allow read-through of premature stop mutations, drugs to assist in break-down of accumulated storage material (cystagon/cysteamine), and even immune suppression therapy (mycophenolate, prednisolone). Enzyme replacement and stem cell therapies have also been evaluated. While many therapeutic approaches have been studied, few have been evaluated in a clinical setting. None are available that slow progression or cure the disease. Patients and families rely on treatments to ameliorate symptoms and palliative care.

The Cln3^{Δex7/8} mouse model was created in the early 2000s to mimic the most common disease-causing mutation in CLN3-Batten disease patients: an approximately 1 kb mutation that eliminates exons 7 and 8 from the CLN3 gene (Cotman et al., Hum Mol Genet. 2002; 11(22):2709-2721; Mole et al., Eur J Paediatr Neurol. 2001; 5:7-10). The mutation is found in a homozygous manner in 85% of the patients and as a heterozygous mutation in combination with point mutations on the other allele in an additional 15% of patients. The exon loss is predicted to produce a frameshift mutation, leading to a truncated protein product with lost or reduced activity (Lerner et al., Cell. 1995 Sep. 22; 82(6):949-57; Kitzmüller et al., Hum Mol Genet. 2008 Jan. 15; 17(2):303-12). In their original study, Cotman et al. demonstrated the CLN3^{Δex7/8} mouse model successfully recapitulated several aspects of CLN3 disease. CLN3^{Δex7/8} animals accumulated autofluorescent storage material and ATP Synthase subunit C in the nervous system at various time points, and exhibited astrocyte reactivity in the brain starting at 10 months of age. Subsequent studies detailed altered glutamate receptor function in the cerebellum, corresponding with motor deficits on an accelerating rotarod assay (Cotman et al., Hum Mol Genet. 2002; 11(22):2709-2721). Behaviorally, Cln3^{Δex7/8} mice have been characterized at both young and mature time points, where neurodevelopmental motor delays were seen in neonatal and young adult mice, and deficits in gait and hind limb clasping were seen at 10-12 months of age (Cotman et al., Hum Mol Genet. 2002; 11(22):2709-2721; Osório et al., Genes Brain Behav. 2009 April; 8(3): 337-345). CLN3^{Δex7/8} mice do not appear to have functional visual impairments, but present with a slight survival deficit when compared to controls at 12 months of age (Cotman et al., Hum Mol Genet. 2002; 11(22):2709-2721; Seigel et al., Mol Cell Neurosci. 2002 April; 19(4):515-27). Taken together, the Cln3^{Δex7/8} mouse model carrying the most frequent human mutation, exhibits numerous cellular and behavioral changes consistent with CLN3-Batten disease, making it a suitable model for testing therapies.

There thus remains a need in the art for treatments for CLN3-Batten Disease.

### SUMMARY

Provided herein are methods and products for CLN3 gene therapy using recombinant AAV.

Provided herein are recombinant adeno-associated virus 9 (rAAV9) encoding a CLN3 polypeptide, comprising an rAAV9 genome comprising in 5′ to 3′ order: a P546 promoter, and a polynucleotide encoding the CLN3 polypeptide. In some embodiments, the rAAV9 genome comprises a self-complementary genome. In some embodiments, the rAAV9 genome comprises a single-stranded genome.

Self-complementary recombinant adeno-associated virus 9 (scAAV9) are provided encoding the CLN3 polypeptide set out in SEQ ID NO: 1, in which the genome of the scAAV9 comprises in 5′ to 3′ order: a first AAV inverted terminal repeat, a P546 promoter comprising the sequence of SEQ ID NO: 3, a polynucleotide encoding the CLN3 polypeptide set out in SEQ ID NO: 1 and a second AAV inverted terminal repeat. The polynucleotide encoding the CLN3 polypeptide may be at least 90% identical to SEQ ID NO: 2.

Also provided are scAAV9 with a genome comprising in 5′ to 3′ order: a first AAV inverted terminal repeat, a P546 promoter comprising the sequence of SEQ ID NO: 3, an SV40 intron, a polynucleotide encoding the CLN3 polypeptide of SEQ ID NO: 1 and a second AAV inverted terminal repeat; scAAV9 with a genome comprising in 5′ to 3′ order: a first AAV inverted terminal repeat, a P546 promoter comprising the sequence of SEQ ID NO: 3, a polynucleotide encoding the CLN3 polypeptide of SEQ ID NO: 1, a bovine growth hormone polyadenylation poly A sequence and a second AAV inverted terminal repeat. In an exemplary embodiment, the scAAV9 has a genome comprising the gene cassette set out in SEQ ID NO: 4.

Single-stranded recombinant adeno-associated virus 9 (ssAAV9) are provided encoding the CLN3 polypeptide set out in SEQ ID NO: 1, in which the genome of the ssAAV9 comprises in 5′ to 3′ order: a first AAV inverted terminal repeat, a P546 promoter comprising the sequence of SEQ ID NO: 3, a polynucleotide encoding the CLN3polypeptide set out in SEQ ID NO: 1 and a second AAV inverted terminal repeat. The polynucleotide encoding the CLN3 polypeptide may be at least 90% identical to SEQ ID NO: 2. Also provided are ssAAV9 with a genome comprising in 5′ to 3′ order: a first AAV inverted terminal repeat, a P546 promoter comprising the sequence of SEQ ID NO: 3, an SV40 intron, a polynucleotide encoding the CLN3 polypeptide of SEQ ID NO: 1 and a second AAV inverted terminal repeat; ssAAV9 with a genome comprising in 5′ to 3′ order: a first AAV inverted terminal repeat, a P546 promoter comprising the sequence of SEQ ID NO: 3, a polynucleotide encoding the CLN3 polypeptide of SEQ ID NO: 1, a bovine growth hormone polyadenylation poly A sequence and a second AAV inverted terminal repeat.

The nucleic acid sequence set out in SEQ ID NO: 4 is the gene cassette that is provided in FIG. 1A. Provided are rAAV9 with an scAAV9 genome or an ssAAV9 genome comprising a nucleic acid sequence that is at least 90% identical to the nucleic acid sequence of SEQ ID NO: 4, or at least 95% identical to the nucleic acid sequence of SEQ ID NO: 4, or at least 98% identical to the nucleic acid sequence of SEQ ID NO: 4.

Nucleic acid molecules comprising a first AAV inverted terminal repeat, a P546 promoter comprising the sequence of SEQ ID NO: 3, a nucleic acid sequence encoding the CLN3 polypeptide of SEQ ID NO: 1 and a second AAV inverted terminal repeat are also provided. In some embodiments, the polynucleotide encoding the CLN3 polypeptide is at least 90% identical to SEQ ID NO: 2.

Also provided are nucleic acid molecules comprising a first AAV inverted terminal repeat, a P546 promoter comprising the nucleotide sequence of SEQ ID NO: 3, an SV40 intron, a nucleic acid sequence encoding the CLN3 polypeptide of SEQ ID NO: 1 and a second AAV inverted terminal repeat. Additionally provided are polynucleotides comprising a first AAV inverted terminal repeat, a P546 promoter comprising the nucleotide sequence of SEQ ID NO: 3, a nucleic acid encoding the CLN3 polypeptide of SEQ ID NO: 1, a bovine growth hormone polyadenylation poly A sequence and a second AAV inverted terminal repeat. In any of the polynucleotides provided, the CLN3 polypeptide may be encoded by the nucleic acid sequence set out in SEQ ID NO: 2 or a sequence at least 90% identical to SEQ ID NO: 2.

rAAV9, scAAV9, or ssAAV9 comprising any of the polynucleotides are provided. rAAV with single-stranded genomes are also provided.

Further provided are rAAV9 viral particles encoding a CLN3 polypeptide, wherein the rAAV9 genome comprises in 5′ to 3′ order: a first AAV inverted terminal repeat, the P546 promoter comprising a nucleic acid sequence at least 90% identical to SEQ ID NO: 3, the polynucleotide encoding a CLN3 polypeptide at least 90% identical to the amino acid sequence of SEQ ID NO: 1, and a second AAV inverted terminal repeat. The rAAV9 particles provided may comprise a polynucleotide encoding the CLN3 polypeptide comprising an amino acid sequence at least 90% identical to SEQ ID NO: 1. In addition, the rAAV9 viral particles may comprise an AAV9 genome comprising a nucleic acid sequence at least 90% identical to the nucleic acid sequence of SEQ ID NO: 4, at least 95% identical to nucleic acid sequence of SEQ ID NO: 4 or at least 98% identical to the nucleic acid sequence of SEQ ID NO: 4. Any of the rAAV9 viral particles may further comprise an SV40 intron, and/or a BGH poly-A sequence.

In any of the rAAV, ssAAV, and the scAAV provided, the AAV inverted terminal repeats may be AAV2 inverted terminal repeats.

Also provided are nucleic acid molecules comprising an rAAV9 genome comprising in 5′ to 3′ order: a first AAV inverted terminal repeat, a P546 promoter comprising a nucleic acid sequence at least 90% identical to SEQ ID NO: 3, and a polynucleotide encoding a CLN3 polypeptide at least 90% identical to the amino acid sequence of SEQ ID NO: 1. The provided nucleic acid molecules may comprise a self-complementary genome or a single-stranded genome.

Further provided are nucleic acid molecules comprising a rAAV9 genome that comprises in 5′ to 3′ order: a first AAV inverted terminal repeat, the P546 promoter comprising a nucleic acid sequence at least 90% identical to SEQ ID NO: 3, the polynucleotide encoding a CLN3 polypeptide at least 90% identical to the amino acid sequence of SEQ ID NO: 1, and a second AAV inverted terminal repeat. The nucleic acid molecules provided may comprise a polynucleotide encoding the CLN3 polypeptide comprising an amino acid sequence at least 90% identical to amino acid sequence of SEQ ID NO: 1. In addition, the nucleic acid molecules may comprise an AAV9 genome comprising a nucleic acid sequence at least 90% identical to the nucleic acid sequence of SEQ ID NO: 4, at least 95% identical to nucleic acid sequence of SEQ ID NO: 4, at least 98% identical to the nucleic acid sequence of SEQ ID NO: 4. Any of the nucleic acid molecules provided may further comprise an SV40 intron, and/or a BGH poly-A sequence.

Further provided are compositions comprising the scAAV9 described herein, the ssAA V9 described herein, nucleic acid molecules described herein or the rAAV viral particles described herein and at least one pharmaceutically acceptable excipient. In some instances, the pharmaceutically acceptable excipient comprises a non-ionic low osmolar compound, a buffer, a polymer, a salt, or a combination thereof. In some embodiments, the polymer is a copolymer. In some embodiments, the copolymer is a poloxamer. For example, the composition may at least comprise a pharmaceutically acceptable excipient comprising a non-ionic, low-osmolar compound. For example, the pharmaceutically acceptable excipient comprises about 20 to 40% non-ionic, low-osmolar compound or about 25% to about 35% non-ionic, low-osmolar compound. An exemplary composition comprises scAAV formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in 1× PBS and 0.001% Polyoxyethylene-polyoxypropylene block copolymer (PLURIONIC® F-68).

Still further provided are methods of treating CLN3-Batten Disease in a subject comprising administering to the subject a composition comprising a therapeutically effective amount of any of the rAAV9 viral particles disclosed herein, any of the scAAV9 disclosed herein, any of the ssAAV9 disclosed herein, any of the nucleic acid molecules described herein or any of the compositions described herein.

In any of the methods provided, the compositions, rAAV9, ssAAV9, scAAV9 and/or nucleic acid molecules are administered via a route selected from the group consisting of intrathecal, intracerebroventricular, intraparenchymal, intravenous, and a combination thereof.

Use of a therapeutically effective amount of any of the rAAV9 viral particles disclosed herein, any of the scAAV9 disclosed herein, any of the ssAAV9 disclosed herein, any of the nucleic acid molecules described herein or any of the compositions described herein for preparation of medicament for treating CLN3-Batten Disease in a subject in need thereof.

Also provided are compositions comprising a therapeutically effective amount of any of the rAAV9 viral particles disclosed herein, any of the scAAV9 disclosed herein, any of the ssAAV9 disclosed herein, any of the nucleic acid molecules described herein or any of the compositions described for treating CLN3-Batten Disease in a subject in need thereof.

Exemplary doses of the scAAV9, ssAAV9, or rAAV9 administered by the intrathecal route are about 1×1011 vg of the scAAV9, ssAAV9, or rAAV9 viral particles subject to about 2×1015 vg per subject, or about 1×1011 vg of the scAAV9, ssAAV9, or rAAV9 viral particles per subject to about 1×1015 vg of the scAAV9, ssAAV9, or AAV9 viral particles per subject, or about 1×1012 vg of the scAAV9, ssAAV9, or rAAV9 viral particles per subject to about 1×1014 vg of the scAAV9, ssAAV9, or AAV9 viral particles per subject, or about 1×1012 vg of the scAAV9, ssAAV9, or rAAV9 viral particles per subject to about 1×1015 vg of the scAAV9, ssAAV9, or AAV9 viral particles per subject. For example, about 1×1013 vg of the scAAV9, ssAAV9, or AAV9 viral particles is administered to a subject, or about 1.5×1013 of the scAAV9, ssAAV9, or AAV9 viral particles is administered to a subject, or about 3.4×1013 of the scAAV9, ssAAV9, or AAV9 viral particles is administered to a subject or about 6×1013 vg of the scAAV9, ssAAV9, or AAV9 viral particles is administered to a subject or about 1.2×1014 of the scAAV9, ssAAV9, or AAV9 viral particles is administered to a subject, or about 2×1014 of the scAAV9, ssAAV9, or AAV9 viral particles is administered to a subject.

The methods, medicaments or compositions for treatment result in a subject, in comparison to the subject before treatment or an untreated CLN3-Batten Disease patient, in one or more of: (a) reduced or slowed lysosomal accumulation of autofluorescent storage material, (b) reduced or slowed lysosomal accumulation of ATP Synthase Subunit C, (c) reduced or slowed glial activation (astrocytes and/or microglia) activation, (d) reduced or slowed astrocytosis, (c) reduced or slowed brain volume loss measured by MRI, (f) reduced or slowed onset of seizures, and (g) stabilization, reduced or slowed progression, or improvement in one or more of the scales that are used to evaluate progression and/or improvement in CLN3 Batten-disease, e.g. the Unified Batten Disease Rating System (UBDRS) assessment scales or the Hamburg Motor and Language Scale. The subject can be held in the Trendelenburg position after administering the rAAV9, ssAAV9, or the scAAV or the nucleic acid molecules disclosed herein.

Still further provided are methods of treating CLN3 disease in a patient in need comprising delivering a composition comprising any one of the rAAV viral disclosed provided herein, any of the scAAV9 disclosed herein, any of the ssAAV9 disclosed herein, any of the nucleic acid molecules described herein, any of the composition described herein or any of the medicaments described herein to a brain or spinal cord of a patient in need thereof.

In any of the methods or uses provided, the composition or medicament may be delivered by intrathecal, intracerebroventricular, intraparenchymal, or intravenous injection of a combination thereof. Any of the methods provided may further comprise placing the patient in the Trendelenburg position after intrathecal injection of the composition, rAAV9 viral particles or the scAAV or the nucleic acid molecules disclosed herein.

In any of the methods or uses provided the compositions or medicaments may comprise a non-ionic, low-osmolar contrast agent. For example, the compositions comprise a non-ionic, low-osmolar contrast agent is selected from the group consisting of iobitridol, iohexol, iomeprol, iopamidol, iopentol, iopromide, ioversol, ioxilan, and combinations thereof.

The compositions or medicaments administered may comprise a pharmaceutically acceptable excipient. For example, the pharmaceutically acceptable excipient comprises about 20 to 40% non-ionic, low-osmolar compound or about 25% to about 35% non-ionic, low-osmolar compound. An exemplary composition comprises scAAV formulated in 20 mM Tris (pH 8.0), 1 mM MgCl2, 200 mM NaCl, 0.001% poloxamer 188 and about 25% to about 35% non-ionic, low-osmolar compound. Another exemplary composition comprises scAAV formulated in 1×PBS and 0.001% Polyoxyethylene-polyoxypropylene block copolymer (PLURIONIC® F-68)

In any of the methods or used provided, when the composition or medicament is delivered to the brain or spinal cord, the composition may be delivered to a brain stem, or may be delivered to the cerebellum, or may be delivered to a visual cortex, or may be delivered to a motor cortex. Further, in any of the methods or uses provided, when the composition or medicament is delivered to the brain or spinal cord, the composition may be delivered to a nerve cell, a glial cell, or both. For example, wherein the delivering to the brain or spinal cord comprises delivery to a cell of the nervous system such as a neuron, a lower motor neuron, a microglial cell, an oligodendrocyte, an astrocyte, a Schwann cell, or a combination thereof.

The methods, uses or administration of the composition or medicament result in a subject, in comparison to the subject before treatment or to an untreated subject, in one or more of: (a) reduced or slowed lysosomal accumulation of autofluorescent storage material, (b) reduced or slowed lysosomal accumulation of ATP Synthase Subunit C, (c) reduced or slowed glial activation (astrocytes and/or microglia) activation, (d) reduced or slowed astrocytosis, (e) reduced or slowed brain volume loss measured by MRI, (f) reduced or slowed onset of seizures, and (g) stabilization, reduced or slowed progression, or improvement in one or more of the scales that are used to evaluate progression and/or improvement in CLN3 Batten-disease, e.g. the Unified Batten Disease Rating System (UBDRS) assessment scales or the Hamburg Motor and Language Scale.

The headings herein are for the convenience of the reader and not intended to be limiting.

The use of ‘may’ and ‘can’ herein is to describe the various embodiments that are included within the claims, and not to indicate uncertainty about the scope of the claims.

### BRIEF DESCRIPTION OF THE DRAWINGS

FIG. 1 provides schematics of the (FIG. 1A) the scAAV9.P546.CLN3 gene cassette and (FIG. 1B) plasmid construct pAAV.P546.CLN3.KAN used for production of scAAV9.P546.CLN3. A human CLN3 cDNA was inserted under the control of the P546 promoter between Inverted Terminal Repeat (ITR) structures derived from AAV2. SV40 Intron (upstream of human CLN3 cDNA) and Bovine Growth Hormone polyadenylation (BGH Poly A) terminator sequence (downstream of human CLN3 cDNA) help in mRNA processing and enhance the transgene expression. The sequence of the plasmid construct pAAV.P546.CLN3.KAN is set out in SEQ ID NO: 5. The genome is packaged in AAV9 capsid protein.

FIG. 2 provides images showing the presence of human CLN3 transcript in CLN3^{Δex7/8} mice injected with scAAV9.P546.CLN3.

FIG. 3 provides graphs showing early reduction in ASM accumulation at 2 months post injection in CLN3^{Δex7/8} mice injected with scAAV9.P546.CLN3. The y-axis on all graphs shown represent total area of ASM accumulation. The black bars represent wild type mice (WT-PBS), the light gray bars represent PBS-injected CLN3^{Δex7/8} mice and the dark gray mice represent scAAV9.P546.CLN3-injected CLN3^{Δex7/8} mice.

FIG. 4 provides graphs for CLN3^{Δex7/8} mice injected with scAAV9.P546.CLN3 show strongly reduced accumulation of ASM at 4 months and 6 months post-injection in various brain regions in PBS-injected wild type mice (“WT”), PBS-injected CLN3^{Δex7/8} (“CLN3”), and scAAV9.P546.CLN3-injected CLN3^{Δex7/8} (“CLN3-AAV”).

FIG. 5A-B provides images and graphs demonstrating that ICV administration of scAAV9.P546.CLN3 reduced the aberrant lysosomal accumulation of the mitochondrial protein ATP Synthase subunit C in the brains of 4 and 6 month old CLN3^{Δex7/8} mice. Representative images of frozen tissue sections stained for ATP synthase subunit C and visualized with DAB staining at the 6 month time point are provided in FIG. 5A. The graphs in FIG. 5B provide the quantification of subunit C accumulation in PBS-injected WT (“WT”), PBS-injected CLN3^{Δex7/8} (“CLN3”), and scAAV9.P546.CLN3-injected CLN3^{Δex7/8} (“CLN3-AAV”) mice at 4 months (4M) and 6 months (6M) post-injection in the somatosensory 1 barrel field of the cortex (SIBF) at 4 and 6 months post-injection and the ventral posteromedial/ventral posterolateral nucleus (VPM/VPL). N=5, p≤0.0001 between untreated CLN3^{Δex7/8} and scAAV9.P546.CLN3 treated animals, as well as wild type animals. p≤0.5 between wild type and treated CLN3^{Δex7/8} mice in the SIBF at 4 and 6 months post-injection.

FIG. 6A-B provides images and graphs for ICV administration of scAAV9.P546.CLN3 reduced astrocytosis in the brains of 4 and 6 month old CLN3^{Δex7/8} mice. FIG. 6A: Representative images (6 month time point) of fixed tissue sections stained for GFAP as a marker for activated astrocytes and visualized with DAB staining. FIG. 6B: Quantification of GFAP-positive arca in PBS-injected WT (“WT”), PBS-injected CLN3^{Δex7/8} (“CLN3”), and scAAV9.P546.CLN3-injected CLN3^{Δex7/8} ( “CLN3-AAV” ) mice at 4 months and 6 months post-injection. N=5 for each group and time point. SIBF=barrel cortex. VPM/VPL=ventral posteromedial/ventral posterolateral nucleus.

FIG. 7A-B provides images and graphs for ICV administration of scAAV9.P546.hCLN3 reduced microglia activation in the brains of 4 and 6 month old CLN3^{Δex7/8} mice. FIG. 7A: Representative images (6 month time point) of fixed tissue sections stained for CD68 as a marker for activated microglia and visualized with DAB staining. FIG. 7B: Quantification of CD68-positive area in PBS injected WT (“WT”), PBS-injected CLN3^{Δex7/8} (“CLN3”), and scAAV9.P546.CLN3-injected CLN3^{Δex7/8} (“CLN3-AAV”) mice at 4 months and 6 months post-injection. N=5 for each group and time point. SIBF=barrel cortex. VPM/VPL=ventral posteromedial/ventral posterolateral nucleus.

FIG. 8 provides graphs showing Rotarod analysis of wild type and PBS or treated CLN3^{Δex7/8} mice up to 18 months post injection. All mice both genders (top panel), male only (middle panel), female only (bottom panel).

FIG. 9 provides graphs showing Morris Water Maze performance of wild type and PBS or scAAV9.P546.CLN3 treated CLN3^{Δex7/8} mice at up to 18 months of age. All mice (top panels), male only (middle panels), female only (bottom panels).

FIG. 10 provides graphs showing performance of scAAV9.P546.CLN3-treated CLN3^{Δex7/8} mice and PBS-treated mice in the pole climbing assay. The pole climbing assay in which mice are place face up on a vertical pole and time for them to turn and descend along with number of falls measures balance and agility. All mice (top panels), male only (middle panels), female only (bottom panels).

FIG. 11 provides graphs showing scAAV9.P546.CLN3-treated CLN3^{Δex7/8} mice fall less often from vertical poles compared to PBS-treated CLN3^{Δex7/8} mice. Mice are place face up on a vertical pole and number of falls measures while attempting to turn around are measured for balance and agility. All mice (top panel), male only (middle panel), female only (bottom panel).

FIG. 12 provides images showing immunofluorescent western blot detection of GFP protein in various brain regions as well as peripheral mouse tissues three weeks post injection with scAAV9.P546.GFP.

FIG. 13 provides a graph showing reverse transcription quantitative PCR of expression of human CLN3 in various brain regions of a four-year old cynomolgus macaques 12 weeks post intrathecal lumbar injection of 3×1013 vg scAAV9.P546.CLN3. The values were normalized to the level of CLN3 protein in the lumbar spinal cord 4-7.

FIG. 14 provides the nucleic acid sequence of scAAV9.P546.CLN3 gene cassette (SEQ ID NO: 4). The AAV2 ITR nucleic acid sequence is in italics (5′ITR is set out as SEQ ID NO: 6 and the 3′ ITR is set out as SEQ ID NO: 9), the P546 promoter nucleic acid sequence (SEQ ID NO: 3) is underlined with a single line, the SV40 intron nucleic acid sequence (SEQ ID NO: 7) is underlined with a double line, the nucleic acid sequence of the human CLN3 cDNA sequence (SEQ ID NO: 2) is in bold, the nucleic acid sequence of the BGH polyA terminator (SEQ ID NO: 8) is underlined with a dotted line.

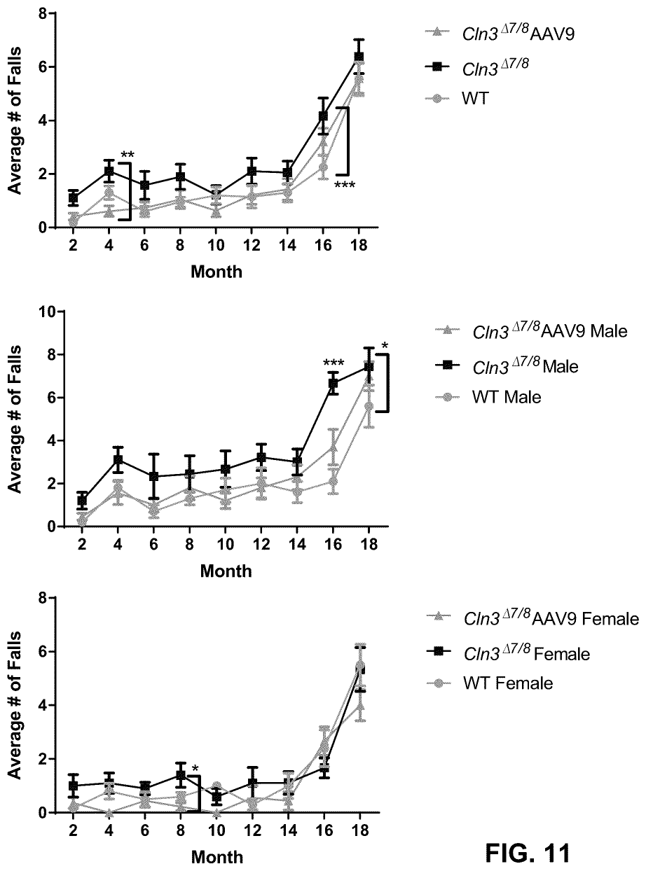

FIG. 15A-B provides the nucleic acid sequence of full length AAV.P546.CLN3 (SEQ ID NO: 5).

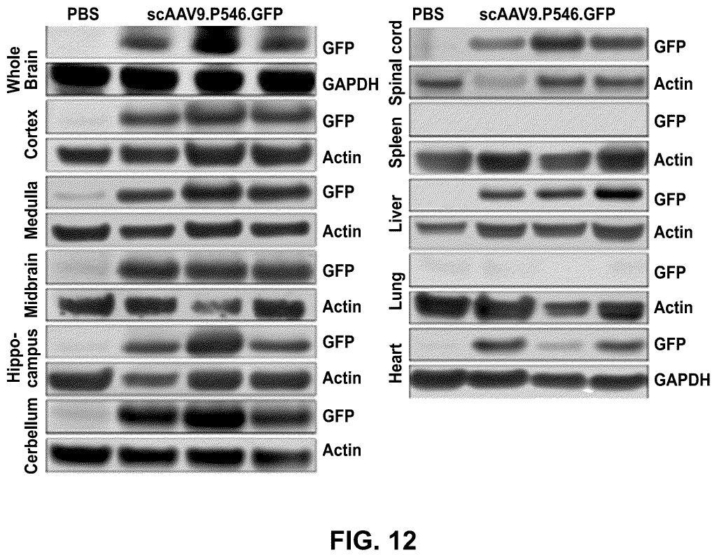

FIG. 16 provides data demonstrating that scAAV9.p546.CLN3 treatment results in increased levels of hCLN3 transcript expression in the cerebral cortex and spinal cord of Cln3^{Δ7/8} mice as measured by qPCR up to 24 months of age. Mean±SEM, ordinary one-way ANOVA at each month, *p<0.05, **p<0.01, ***p<0.001, ****p<0.0001

FIG. 17 provides images showing that scAAV9.p546.CLN3 treatment produces stable hCLN3 transcript throughout the brain of Cln3^{Δ7/8} mice as measured by in situ hybridization (RNASCOPE®) (red fluorescence), up to 24 months of age. Images taken at 20×.

FIG. 18 provides data demonstrating that scAAV9.p546.CLN3 treatment prevented and reduced ASM accumulation in two areas of the brain in Cln3^{Δ7/8} mice up to 24 months of age. Mean±SEM, ordinary one-way ANOVA at each month, *p<0.05, **p<0.01, ***p<0.001, ****p<0.0001. Images taken at 20×.

FIG. 19 provides data demonstrating that scAAV9.p546.CLN3 treatment prevented large amounts of SubUnitC accumulation, (a constituent of the ASM) in two areas of the brain of Cln3^{Δ7/8} mice up to 24 months of age. Mean±SEM, ordinary one-way ANOVA at each month, *p<0.05, **p<0.01, ***p<0.001, ****p<0.0001. Images taken at 20×.

FIG. 20 provides data demonstrating that scAAV9.p546.CLN3 treatment prevented astrocyte activation (GFAP⁺) in two areas of the Cln3^{Δ7/8} brain up to 24 months of age. Mean±SEM, ordinary one-way ANOVA at each month, *p<0.05, **p<0.01, ***p<0.001, ****p<0.0001. Images taken at 20×.

FIG. 21 provides data demonstrating scAAV9.p546.CLN3 treatment prevented some microglial activation (CD68⁺) in the two areas of the Cln3^{Δ7/8} brain up to 24 months of age, dependent on time point. Mean±SEM, ordinary one-way ANOVA at each month, *p<0.05, **p<0.01, ***p<0.001, ****p<0.0001. Images taken at 20×

FIG. 22 provides data demonstrating scAAV9.CB.CLN3 treatment is similarly effective in preventing various Batten disease pathologies in 6 and 12 month old Cln3^{Δ7/8} mice. Mean±SEM, ordinary two-way ANOVA, *p<0.05, **p<0.01, ***p<0.001, ****p<0.0001.

FIG. 23 provides data demonstrating Cln3^{Δ7/8} have no red blood cell abnormalities, as measured up to 24 months of age. Mean±SEM, ordinary two-way ANOVA

FIG. 24 provides data Cln3^{Δ7/8} mice have no white blood cell abnormalities, as measured up to 24 months of age. Mean±SEM, ordinary two-way ANOVA

FIG. 25 demonstrates scAAV9.p546.CLN3 treated mice show differing levels of SubC accumulation in the CA3 region of the hippocampus based on sex at 12 months of age. Mean±SEM, Ordinary Two-way ANOVA with Tukey's post-hoc test, *p<0.05, **p<0.01, ***p<0.001, ****p<0.0001.

FIG. 26 demonstrates scAAV9.p546.CLN3 mice show subtle, differing levels of SubC accumulation in the Piriform Cortex (PIRC) based on sex at multiple time points. Mean±SEM, Ordinary Two-way ANOVA with Tukey's post-hoc test, *p<0.05, **p<0.01, ***p<0.001, ****p<0.0001.

FIG. 27 demonstrates scAAV9.p546.CLN3 mice show differing levels of SubC accumulation in the Reticular Thalamic Nucleus (RTN) based on sex at multiple time points. Mean±SEM, Ordinary Two-way ANOVA with Tukey's post-hoc test, *p<0.05, **p<0.01, ***p<0.001, ****p<0.0001.

FIG. 28 demonstrates scAAV9.p546.CLN3 mice show differing levels of SubC accumulation in the Somatosensory Cortex based on sex at 12 months. Mean±SEM, Ordinary Two-way ANOVA with Tukey's post-hoc test, *p<0.05, **p<0.01, ***p<0.001, ****p<0.0001.

FIG. 29 demonstrates scAAV9.p546.CLN3 mice show differing levels of SubC accumulation in the VPM/VPL of the thalamus based on sex at 12 months. Mean±SEM, Ordinary Two-way ANOVA with Tukey's post-hoc test, *p<0.05, **p<0.01, ***p<0.001, *p<0.0001.

FIG. 30 demonstrates scAAV9.p546.CLN3 mice show differing levels of SubC accumulation in the Basolateral Amygdala (BLA) based on sex at 12 months. Mean±SEM, Ordinary Two-way ANOVA with Tukey's post-hoc test, *p<0.05, **p<0.01, ***p<0.001, ****p<0.0001.

FIG. 31 provides scAAV9.p546.CLN3 mice show differing levels of SubC accumulation in the Polymorphic Layer of the Dentate Gyrus (DG) based on sex at 12 and 18 months. Mean±SEM, Ordinary Two-way ANOVA with Tukey's post-hoc test, *p<0.05, **p<0.01, ***p<0.001, ****p<0.0001.

FIG. 32 demonstrates scAAV9.p546.CLN3 mice show differing levels of SubC accumulation in the Habenula (Hab) based on sex at 12 and 18 months. Mean±SEM, Ordinary Two-way ANOVA with Tukey's post-hoc test, *p<0.05, **p<0.01, ***p<0.001, ** **p<0.0001.

FIG. 33 provides data demonstrating scAAV9.p546.CLN3 mice show differing levels of SubC accumulation in the Mediodorsal Nucleus (MD) based on sex at 12 and 18 months. Mean±SEM, Ordinary Two-way ANOVA with Tukey's post-hoc test, *p<0.05, **p<0.01, ***p<0.001, ****p<0.0001.

FIG. 34 demonstrates that scAAV9.p546.CLN3 mice show no difference in levels of SubC accumulation in the Retrosplenial Cortex (RSC) based on sex. Mean±SEM, Ordinary Two-way ANOVA with Tukey's post-hoc test, *p<0.05, **p<0.01, ***p<0.001, ****p<0.0001.

FIG. 35 demonstrates that scAAV9.p546.CLN3 mice show differing levels of activated microglia (CD68⁺) in the Somatosensory Cortex (SIBF) based on sex at 6 months. Mean±SEM, Ordinary Two-way ANOVA with Tukey's post-hoc test, *p<0.05, **p<0.01, ***p<0.001, ****p<0.0001.

FIG. 36 demonstrates that scAAV9.p546.CLN3 mice show differing levels of microglia (CD68⁺) activation in the VPM-VPL, thalamus based on sex. Mean±SEM, Ordinary Two-way ANOVA with Tukey's post-hoc test, *p<0.05, **p<0.01, ***p<0.001, ****p<0.0001.

FIG. 37 demonstrates that scAAV9.p546.CLN3 mice show differing levels of activated microglia (CD68⁺) in the Mediodorsal Nucleus (MD) based on sex. Mean±SEM, Ordinary Two-way ANOVA with Tukey's post-hoc test, *p<0.05, **p<0.01, ***p<0.001, p<0.0001.

FIG. 38 demonstrates that scAAV9.p546.CLN3 mice show differing levels of activated microglia (CD68⁺) in the Submedial Nucleus (SM) based on sex. Mean±SEM, Ordinary Two-way ANOVA with Tukey's post-hoc test, *p<0.05, **p<0.01, ***p<0.001, ****p<0.0001.

### DETAILED DESCRIPTION

The present disclosure provides methods and products for treating CLN3-Batten Disease. The methods involve delivery of a CLN3 polynucleotide to a subject using rAAV as a gene delivery vector.

Adeno-associated virus (AAV) is a replication-deficient parvovirus, the single-stranded DNA genome of which is about 4.7 kb in length including 145 nucleotide inverted terminal repeat (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific clade, the members of which share serologic and functional similarities. Thus, AAVs may also be referred to by the clade. For example, AAV9 sequences are referred to as “clade F” sequences (Gao et al., J. Virol., 78: 6381-6388 (2004). The present disclosure contemplates the use of any sequence within a specific clade, e.g., clade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-BI genome is provided in Choudhury et al., Mol. Ther., 24(7): 1247-1257 (2016). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The native AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. In some instances, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV,” “AAV virus,” and “AAV viral particle” are used interchangeably herein. The term “rAAV” refers to a recombinant AAV virus or recombinant infectious, encapsulated viral particles. The terms “rAAV,” “rAAV virus,” and “rAAV viral particle” are used interchangeably herein.

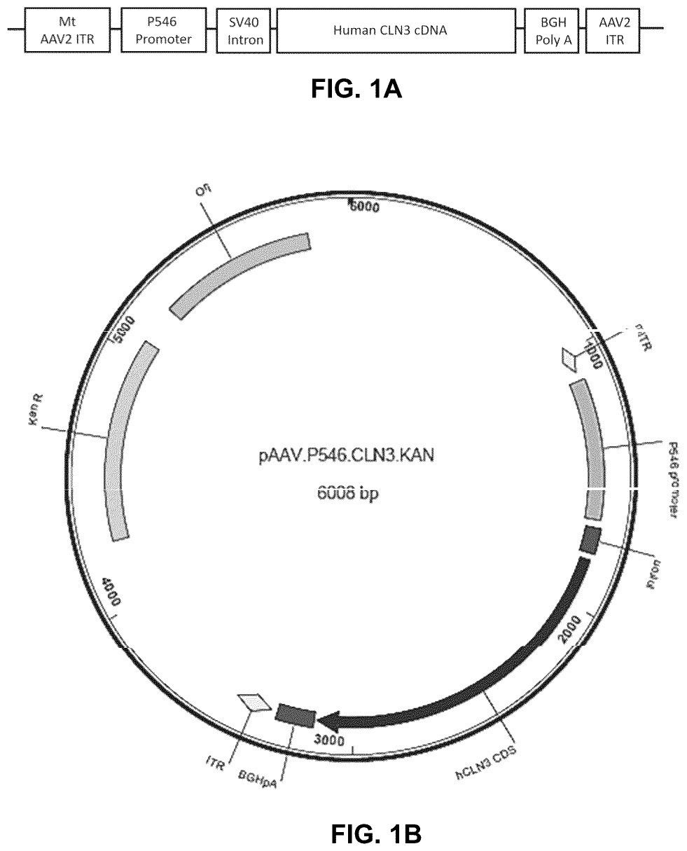

The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest (e.g., a CLN3-encoding polynucleotide) flanked on the 5′ and 3′ ends by inverted terminal repeat (ITR). In some embodiments, the rAAV genome comprises a “gene cassette.” An exemplary gene cassette is set out in FIG. 1A and the nucleic acid sequence of SEQ ID NO: 4. The rAAV genome can be a self-complementary (sc) genome, which is referred to herein as “scAAV genome.” Alternatively, the rAAV genome can be a single-stranded (ss) genome, which is referred to herein as “ssAAV genome.”

The term “scAAV” refers to a rAAV virus or rAAV viral particle comprising a self-complementary genome. The term “ssAAV” refers to a rAAV virus or rAAV viral particle comprising a single-stranded genome.

rAAV genomes provided herein may comprise a polynucleotide encoding a CLN3 polypeptide. CLN3 polypeptides comprise the amino acid sequence set out in SEQ ID NO: 1, or a polypeptide with an amino acid sequence that is at least: 90%, 91%, 92%, 93%, 94%, 95%, 96%, 97%, 98%, or 99% identical to the amino acid sequence set forth in SEQ ID NO: 1, and which encodes a polypeptide with CLN3 activity (e.g., at least one of increasing clearance of lysosomal auto-fluorescent storage material, reducing lysosomal accumulation of ATP synthase subunit C, and reducing activation of astrocytes and microglia in a patient when treated as compared to, e.g. the patient prior to treatment).

rAAV genomes provided herein, in some cases, comprise a polynucleotide encoding a CLN3 polypeptide wherein the polynucleotide has the nucleotide sequence set out in SEQ ID NO: 2, or a polynucleotide at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 2 and encodes a polypeptide with CLN3 activity (e.g., at least one of increasing clearance of lysosomal auto-fluorescent storage material, reducing lysosomal accumulation of ATP synthase subunit C, and reducing activation of astrocytes and microglia in a patient when treated as compared to, e.g. the patient prior to treatment).

rAAV genomes provided herein, in some embodiments, comprise a polynucleotide sequence that encodes a polypeptide with CLN3 activity and that hybridizes under stringent conditions to the nucleic acid sequence of SEQ ID NO: 2, or the complement thereof. The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing include but are not limited to 0.015 M sodium chloride, 0.0015 M sodium citrate at 65-68° C. or 0.015 M sodium chloride, 0.0015M sodium citrate, and 50% formamide at 42° C. Sec, for example, Sambrook et al., Molecular Cloning: A Laboratory Manual, 2nd Ed., Cold Spring Harbor Laboratory, (Cold Spring Harbor, N.Y. 1989).

The rAAV genomes provided herein, in some embodiments, comprise one or more AAV ITRs flanking the polynucleotide encoding a CLN3 polypeptide. The CLN3 polynucleotide is operatively linked to transcriptional control elements (including, but not limited to, promoters, enhancers and/or polyadenylation signal sequences) that are functional in target cells to form a gene cassette. Examples of promoters are the chicken ß actin promoter and the P546 promoter. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Additionally provided herein are a P546 promoter sequence set out in SEQ ID NO: 3, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 3 that are promoters with P546 transcription promoting activity. Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a CLN3 RNA transcript when expressed in mammalian cells. One example of such an intron is the SV40 intron. “Packaging” refers to a series of intracellular events that result in the assembly and encapsidation of an AAV particle. The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allows AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV rh.74 and AAV-B1. As noted above, the nucleotide sequences of the genomes of various AAV serotypes are known in the art. rAAV with capsid mutations are also contemplated. See, for example, Marsic et al., Molecular Therapy, 22(11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. Sec, for example, Giles et al., Molecular Therapy, 26(12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids may be transferred to cells permissible for infection with a helper virus of AAV (e.g., adenovirus, El-deleted adenovirus or herpesvirus) for assembly of the rAAV genome into infectious viral particles with AAV9 capsid proteins. Techniques to produce rAAV, in which an rAAV genome to be packaged, rep and cap genes, and helper virus functions are provided to a cell are standard in the art. Production of rAAV particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. In various embodiments, AAV capsid proteins may be modified to enhance delivery of the recombinant rAAV. Modifications to capsid proteins are generally known in the art. Sec, for example, US 2005/0053922 and US 2009/0202490, the disclosures of which are incorporated by reference herein in their entirety.

A method of generating a packaging cell is to create a cell line that stably expresses all the necessary components for rAAV production. For example, a plasmid (or multiple plasmids) comprising a rAAV genome lacking AAV rep and cap genes, AAV rep and cap genes separate from the rAAV genome, and a selectable marker, such as a neomycin resistance gene, may be integrated into the genome of a cell. rAAV genomes may be introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line may then be infected with a helper virus such as adenovirus. The advantages of this method are that the cells are selectable and are suitable for large-scale production of rAAV. Other non-limiting examples of suitable methods employ adenovirus or baculovirus rather than plasmids to introduce rAAV genomes and/or rep and cap genes into packaging cells.

General principles of rAAV particle production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV particle production.

Further provided herein are packaging cells that produce infectious rAAV particles. In one embodiment packaging cells may be stably transformed cancer cells such as HeLa cells, 293 cells and PerC.6 cells (a cognate 293 line). In another embodiment, packaging cells may be cells that are not transformed cancer cells such as low passage 293 cells (human fetal kidney cells transformed with El of adenovirus), MRC-5 cells (human fetal fibroblasts), WI-38 cells (human fetal fibroblasts), Vero cells (monkey kidney cells) and FRhL-2 cells (rhesus fetal lung cells).

Also provided herein are rAAV (e.g., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. A rAAV with a single-stranded genome is referred to herein as an ssAAV.

An exemplary rAAV provided herein is the scAAV named “scAAV9.P546.CLN3.” The scAAV9.P546.CLN3 scAAV contains a scAAV genome comprising a human CLN3 cDNA under the control of a P546 promoter (SEQ ID NO: 3). The scAAV genome also comprises a SV40 Intron (upstream of human CLN3 cDNA) and Bovine Growth Hormone polyadenylation (BGH Poly A) terminator sequence (downstream of human CLN3 cDNA). The sequence of this scAAV9.P546.CLN3 gene cassette is set out in SEQ ID NO: 4. The scAAV genome is packaged in an AAV9 capsid and includes AAV2 ITRs (one ITR upstream of the P546 promoter and the other ITR downstream of the BGH Poly A terminator sequence).

The rAAV may be purified by methods standard in the art such as by column chromatography or cesium chloride gradients. Methods for purifying rAAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

Compositions comprising rAAV are also provided. Compositions comprise an rAAV encoding a CLN3 polypeptide. Compositions may include two or more rAAV encoding different polypeptides of interest. In some embodiments, the rAAV is scAAV or SSAAV.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Polyoxyethylene-polyoxypropylene block copolymer (PLURIONIC® F-68)) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgI/mL, an osmolality by vapor-pressure osmometry of about 322mOsm/kg water, an osmolarity of about 273mOsm/L, an absolute viscosity of about 2.3cp at 20° C. and about 1.5cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in and 1× PBS and 0.001% Polyoxyethylene-polyoxypropylene block copolymer (PLURIONIC® F-68).

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include about 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.4×10¹³, about 3.5×10¹³, about 4×10¹³, about 4.5×10¹³, about 5×10¹³, about 6×10¹³, about 1×10¹⁴, about 1.2×10¹⁴, about 2×10¹⁴, about 3×10¹⁴, about 4×10¹⁴ about 5×10¹⁴, about 1×10¹⁵, to about 1×10¹⁶, or more total viral genomes. Dosages of about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, and about 6×10¹³ to about 1.2×10¹⁴ vg are also contemplated. One dose exemplified herein is 6×10¹³ vg. Another dose exemplified herein is 1.2×10¹⁴.

Methods of transducing target cells (including, but not limited to, cells of the nervous system, nerve or glial cells) with rAAV are provided. The cells of the nervous system include neurons, lower motor neurons, microglial cells, oligodendrocytes, astrocytes, Schwann cells or combinations thereof.

The term “transduction” is used to refer to the administration/delivery of the CLN3 polynucleotide to a target cell either in vivo or in vitro, via a replication-deficient rAAV of the disclosure resulting in expression of a functional polypeptide by the recipient cell. Transduction of cells with rAAV of the disclosure results in sustained expression of polypeptide or RNA encoded by the rAAV. The present disclosure thus provides methods of administering/delivering to a subject rAAV encoding a CLN3 polypeptide by an intrathecal, intracerebroventricular, intraparennchymal, or intravenous route, or any combination thereof. Intrathecal delivery refers to delivery into the space under the arachnoid membrane of the brain or spinal cord. In some embodiments, intrathecal administration is via intracisternal administration.

Intrathecal administration is exemplified herein. These methods include transducing target cells (including, but not limited to, nerve and/or glial cells) with one or more rAAV described herein. In some embodiments, the rAAV viral particle comprising a polynucleotide encoding a CLN3 polypeptide is administered or delivered the brain and/or spinal cord of a patient. In some embodiments, the polynucleotide is delivered to brain. Areas of the brain contemplated for delivery include, but are not limited to, the motor cortex, visual cortex, cerebellum and the brain stem. In some embodiments, the polynucleotide is delivered to the spinal cord. In some embodiments, the polynucleotide is delivered to a neuron or lower motor neuron. The polynucleotide may be delivered to nerve and glial cells. The glial cell is a microglial cell, an oligodendrocyte or an astrocyte. In some embodiments, the polynucleotide is delivered to a Schwann cell.

In some embodiments of methods provided herein, the patient is held in the Trendelenburg position (head down position) after administration of the rAAV (e.g., for about 5, about 10, about 15 or about 20 minutes). For example, the patient may be tilted in the head down position at about 1 degree to about 30 degrees, about 15 to about 30 degrees, about 30 to about 60 degrees, about 60 to about 90 degrees, or about 90 to about 180 degrees).

The methods provided herein comprise the step of administering an effective dose, or effective multiple doses, of a composition comprising a rAAV provided herein to a subject (e.g., an animal including, but not limited to, a human patient) in need thereof. If the dose is administered prior to development of CLN3-Batten Disease, the administration is prophylactic. If the dose is administered after the development of CLN3-Batten Disease, the administration is therapeutic. An effective dose is a dose that alleviates (eliminates or reduces) at least one symptom associated with the disease, that slows or prevents progression of the disease, that diminishes the extent of disease, that results in remission (partial or total) of disease, and/or that prolongs survival. In comparison to the subject before treatment or in comparison to an untreated subject, methods provided herein result in stabilization, reduced progression, or improvement in one or more of the scales that are used to evaluate progression and/or improvement in CLN3 Batten-disease, e.g. the Unified Batten Disease Rating System (UBDRS) or the Hamburg Motor and Language Scale. The UBDRS assessment scales (as described in Marshall et al., Neurology. 2005 65(2):275-279) [including the UBDRS physical assessment scale, the UBDRS seizure assessment scale, the UBDRS behavioral assessment scale, the UBDRS capability assessment scale, the UBDRS sequence of symptom onset, and the UBDRS Clinical Global Impressions (CGI)]; the Pediatric Quality of Life Scale (PEDSQOL) scale, motor function, language function, cognitive function, and survival. In comparison to the subject before treatment or in comparison to an untreated subject, methods provided herein may result in one or more of the following: reduced or slowed lysosomal accumulation of autofluorescent storage material, reduced or slowed lysosomal accumulation of ATP Synthase Subunit C, reduced or slowed glial activation (astrocytes and/or microglia) activation; reduced or slowed astrocytosis, and showed a reduction or delay in brain volume loss measured by MRI.

Combination therapies are also provided. Combination, as used herein, includes either simultaneous treatment or sequential treatment. Combinations of methods described herein with standard medical treatments are specifically contemplated. Further, combinations of compositions (e.g., a combination of scAAV9.P546.CLN3 and a contrast agent disclosed herein) for use according to the invention—either simultaneous treatment or sequential treatment—are specifically contemplated.

While delivery to a subject in need thereof after birth is contemplated, intrauterine delivery to a fetus is also contemplated.

### EXAMPLES

While the following examples describe specific embodiments, it is understood that variations and modifications will occur to those skilled in the art. Accordingly, only such limitations as appear in the claims should be placed on the invention.

In the examples, a self-complementary AAV (named scAAV9.P546.CLN3) carrying a CLN3 cDNA under the control of a P546 promoter was produced. The P546 promoter is a truncated version of the MeCP2 promoter, allowing expression of the transgene in both neurons and astrocytes at moderate levels. The efficacy of this gene therapy vector was tested in the CLN3^{Δex7/8} knock-in mouse model, which carries the most frequent mutation found in human patients. The safety and efficacy of scAAV9.P546.CLN3 were evaluated in vivo in the CLN3^{Δex7/8} knock-in mouse model, wild type mice and non-human primates. Data from mice and non-human primates clearly demonstrate efficient transduction of astrocytes and neurons throughout the brain and spinal cord including deep brain structures, such as thalamus, hippocampus, striatum, amygdala, medulla and cerebellum.

### Example 1

### Production of scAAV9.P546.CLN3

A DNA including the open reading frame of human CLN3 (SEQ ID NO: 2) between two Not1 restriction sites was synthesized by Eurofin Genomics, USA, and then inserted in a double-stranded AAV2-ITR-based production plasmid. A schematic of the plasmid construct showing the CLN3 DNA inserted between AAV2 ITRs [the 5′ ITR was modified as previously described in McCarty et al., Gene Therapy 8:1248-1254 (2001) to generate scAAV] is shown in FIG. 1. The plasmid construct also includes the P546 promoter, an SV40 chimeric intron and a Bovine Growth Hormone (BGH) polyadenylation signal.

scAAV9.P546.CLN3 was produced under cGMP conditions by transient triple-plasmid transfection procedures using the double-stranded AAV2-ITR-based production plasmid, with a plasmid encoding Rep2Cap9 sequence as previously described [Gao et al., J. Virol., 78: 6381-6388 (2004)], along with an adenoviral helper plasmid pHelper (Stratagene, Santa Clara, CA) in HEK293 cells. Virus was purified by two cesium chloride density gradient purification steps, dialyzed against PBS and formulated with 0.001% Polyoxyethylene-polyoxypropylene block copolymer (PLURIONIC® F-68) to prevent virus aggregation and stored at 4° C. All scAAV preparations were titered by quantitative PCR using hydrolysis probes (TAQMAN® technology). Purity of scAAVwas assessed by 4-12% sodium dodecyl sulfate-acrylamide gel electrophoresis and silver staining (Invitrogen, Carlsbad, CA).

### Example 2

### Long-Term Efficacy Study of CSF-Delivered scAAV9.P546.CLN3 in CLN3^{Δex7/8} Mice

### Cell Targeting and Expression

To confirm the expression and biodistribution of virally-introduced human CLN3 in mice, scAAV9.P546.CLN3 was formulated in 1× PBS and 0.001% Polyoxyethylene-polyoxypropylene block copolymer (PLURIONIC® F-68) or formulated in 20 mM Tris (pH8.0), 1 mM MgCl2, 200 mM NaCl, 0.001%. poloxamer 188, and administered into CLN3^{Δex7/8} mice via intracerebroventricular (ICV) injection within 36 hours of birth and expression was monitored at various time points. Wild type and CLN3^{Δex7/8} mice injected with an equal volume of PBS served as controls. The effective administered dose was 2.2×10¹⁰ vg/mouse using the NCH viral vector core titer.

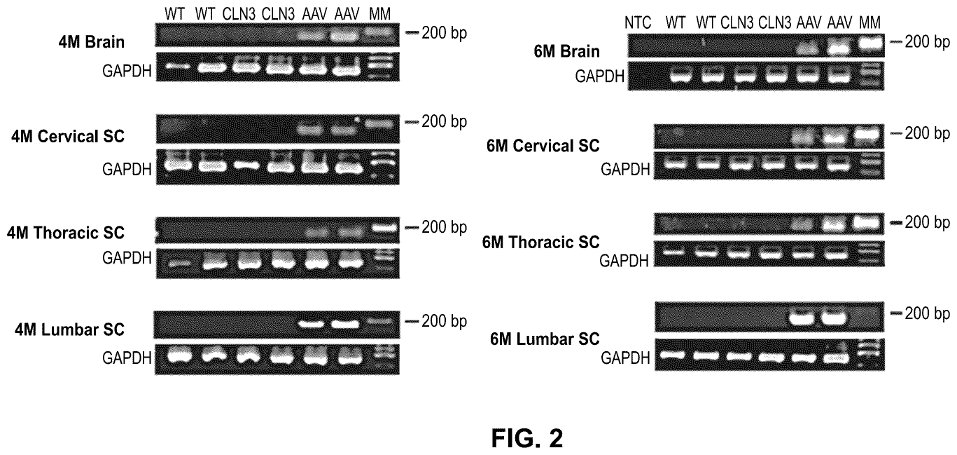

To obtain a detailed brain biodistribution profile, in situ hybridization techniques (RNASCOPE®) were used to specifically identify human CLN3 mRNA in the brain, cervical spinal cord, thoracic spinal cord and the lumbar spinal cord. This technique involved using RNA in situ hybridization with specific probes to detect only the human transgene encoded by the scAAV9. A strong signal was observed at 4 months and 6 months post-injection, particularly in the cortex (regions A-C) of CLN3^{Δex7/8} mice injected with scAAV9.P546.CLN3 compared to no signal in PBS injected controls. The analysis demonstrated that the AAV9 delivered CLN3 transgene was expressed at adequate levels in various regions of the brain including cortex, thalamus, hindbrain, cerebellum and spinal cord. In the cerebellum, the signal was particularly strong in Purkinje neurons. Transgene expression was also detected in brain and all regions of the spinal cord via reverse transcription PCR at 4 and 6 months (FIG. 2).

Taken together, the reverse transcription PCR data and in situ hybridization (RNASCOPE®) analysis performed on tissue from CLN3^{Δex7/8} mice, confirmed that a single ICV injection of scAAV9.P546.CLN3 led to successful targeting and expression of human CLN3 throughout the brain and spinal cord up to 6 months post injection. This confirms the validity of ICV-mediated delivery of the scAAV9to specifically target cells that are disproportionately involved in the pathogenesis of CLN3-Batten disease. The expression data in the CLN3^{Δex7/8} mouse model were further confirmed in studies in wild type mice using the same primers for detection of the human transgene via quantitative RT-PCR.

Pathology Improvements Following Delivery of scAAV9.P546.CLN3

### Accumulation of Autofluorescent Storage Material (ASM)

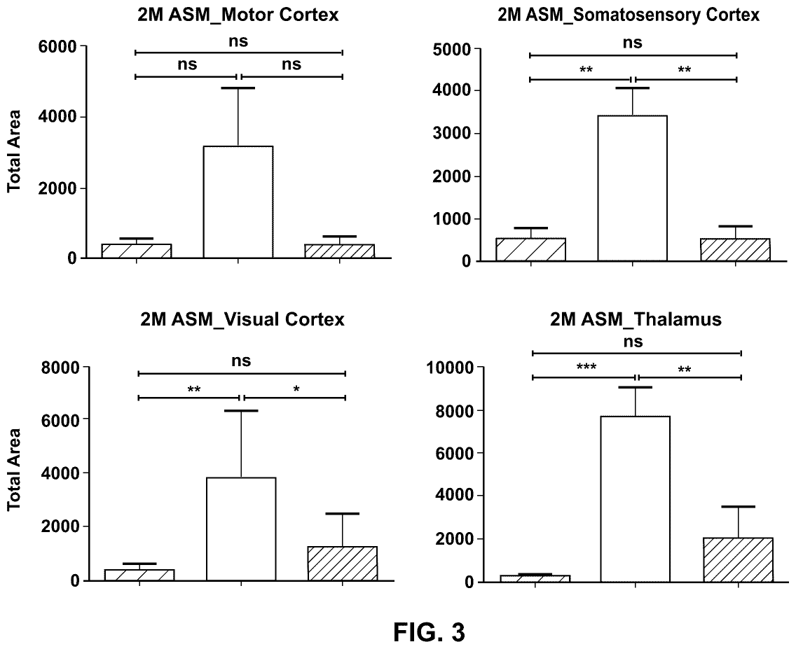

Accumulation of autofluorescent storage material (ASM) is the hallmark histological marker for Batten disease progression (Mole et al., Biochim Biophys Acta-Mol Basis Dis. 2015; 1852(10):2237-2241; Cotman et al., Clin Lipidol. 2012 February; 7(1):79-91; Seehafer et al., Neurobiol Aging. 2006; 27:576-588). Accumulation of ASM is a strong indicator for disease progression for many forms of Batten disease (Bosch et al., J Neurosci. 2016; 36(37):9669-9682; Morgan et al., PLOS One. 2013; 8(11):e78694). It is contemplated herein that reduction of ASM is used as indicator of successful treatment. Being one of the earliest detectable disease manifestations of CLN3-Batten disease, ASM was already visible in numerous brain regions of CLN3^{Δex7/8} mice by 2 months of age (FIG. 3).

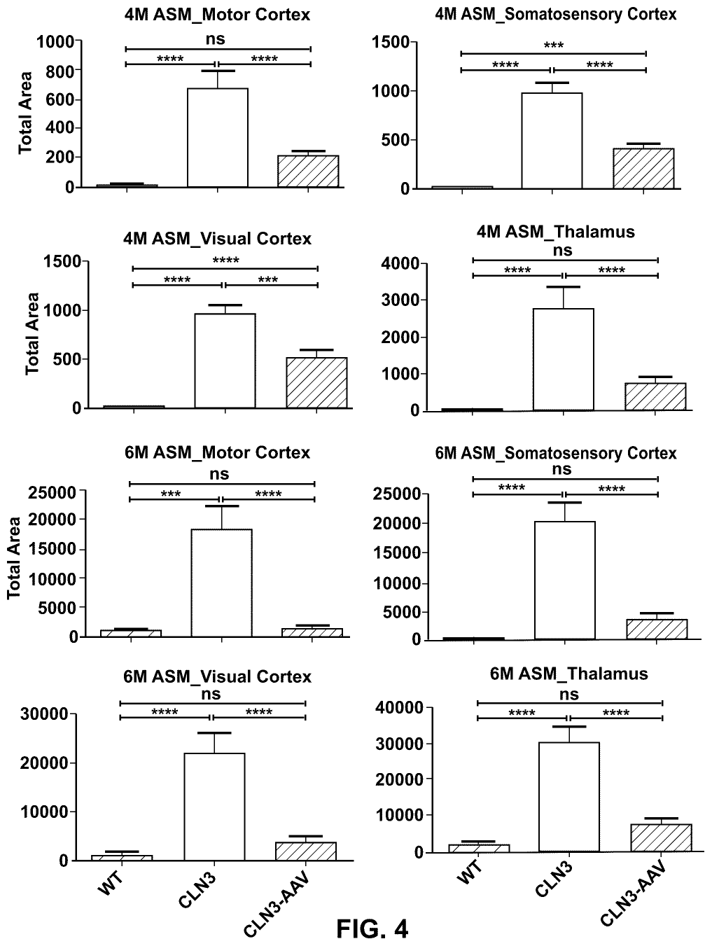

Automated quantification of fluorescent pixel area confirmed a significant reduction in accumulated ASM in the somatosensory cortex and thalamus in scAAV9.P546.CLN3-injected CLN3^{Δex7/8} mice at 2 months of age. Since at this early time point, higher variability of ASM accumulation in motor cortex and visual cortex were seen in PBS treated CLN3^{Δex7/8} mice, the statistical power of the analysis was lower for these two areas. At 4 and 6 months post injection, all four brain areas showed highly significant reduction in ASM accumulation compared to PBS treated CLN3^{Δex7/8} mice (FIG. 4). When comparing scAAV9.P546.CLN3-injected CLN3^{Δex7/8} mice to wild type animals, slightly higher ASM levels were found in the somatosensory and visual cortex of scAAV9.P546.CLN3-injected CLN3^{Δex7/8} mice at 4 months post injection, while no significant difference was found between wild type and scAAV9.P546.CLN3 treated CLN3^{Δex7/8} mice in motor cortex and thalamus. At 6 months post injection, all areas showed comparable low levels of ASM in wild type and scAAV9.P546.CLN3 treated CLN3^{Δex7/8} mice that were much lower compared to PBS treated CLN3^{Δex7/8} mice, confirming the long lasting and highly efficient reduction in ASM accumulation (p≤0.0001). (N=10 per group), p≤0.0001 for all but visual cortex at 4 months (p≤0.001) and motor cortex (p≤0.001) at 6 months post injection between PBS and scAAV9.P546.CLN3 treated animals.

### Accumulation of Mitochondrial Protein ATP Synthase Subunit C

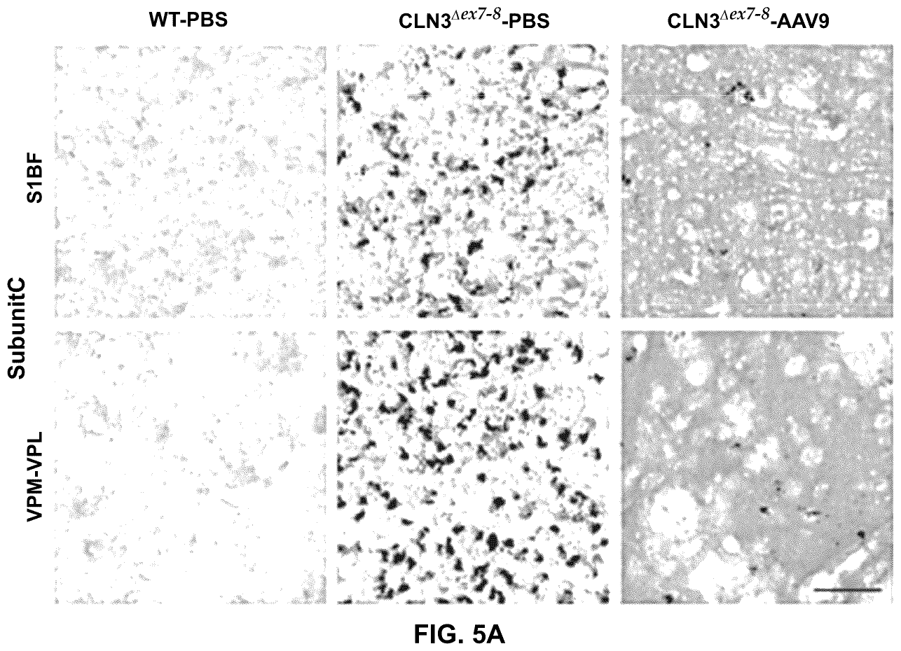

Brain tissue of wild type and CLN3^{Δex7/8} PBS-injected or scAAV9.P546.CLN3-injected mice was analyzed for accumulation of ATP synthase subunit C. In healthy individuals, this protein is part of the respiratory chain in the mitochondrial membrane, but in patients suffering from Batten disease, the protein aberrantly accumulates in lysosomes (Palmer et al., Am J Med Genet. 1992; 42(4):561-567). In the CLN3^{Δex7/8} mouse, compared to wild type animals, subunit C accumulation is apparent by 4 months of age in the ventral posteromedial nucleus and ventral posterolateral nucleus of the thalamus (VPM/VPL region), a brain region often affected early on in NCL mouse models (Morgan et al., PLOS One. 2013; 8(11):c78694; Pontikis et al., Neurobiol Dis. 2005; 20(3):823-836). While untreated animals showed a strong signal for accumulated ATP synthase sub C in the somatosensory cortex and the VPM/VPL area of the thalamus, scAAV9.P546.CLN3 treated animals displayed minimal signal that was comparable to wild type animals at both, 4 and 6 months post injection (FIG. 5A-B) (p≤ 0.0001 between PBS and scAAV9.P546.CLN3 treated animals).

### Glial and Astrocyte Activation

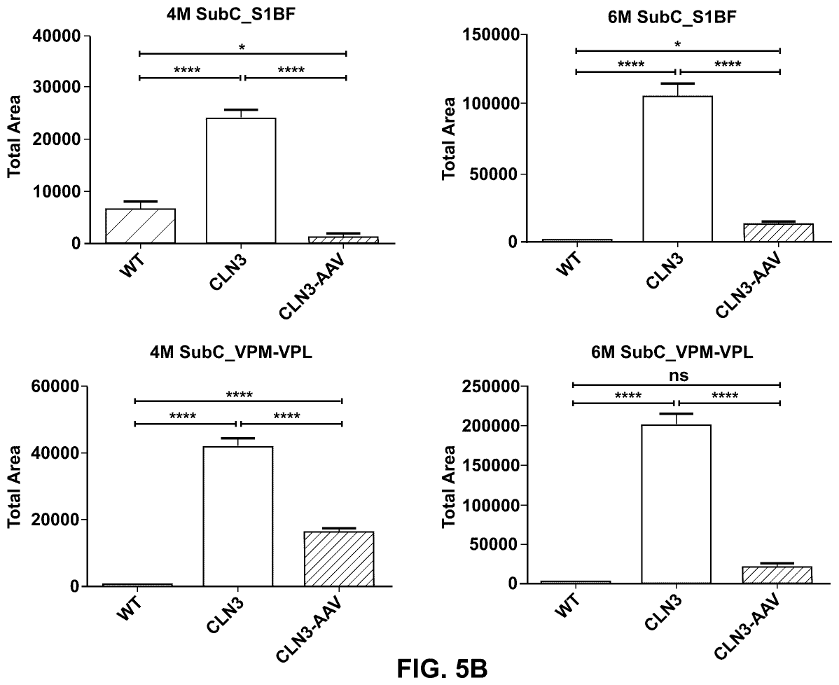

Besides aberrant accumulation of storage material and accumulation of ATP Synthase sub C, other histological markers of disease progression in both human patients and animal models include activation of astrocytes and microglia (Cotman et al., Hum Mol Genet. 2002; 11(22):2709-2721; Morgan et al., PLOS One. 2013; 8(11):e78694; Pontikis et al., Neurobiol Dis. 2005; 20(3):823-836; Palmer et al., Am J Med Genet. 1992; 42(4):561-567). In particular, reactive microglia are primed to release pro-inflammatory mediators such as IL1-β26, which may be a key contributing cause of neuronal cell death at the later stages of CLN3-Batten disease. Activated astrocytes were identified in VPM/VPL thalamus and somatosensory cortex sections by staining for glial fibrillary acidic protein (GFAP) at 4 and 6 month timepoints. For the somatosensory cortex, quantification was performed in the barrel cortex within cortical layer IV of the somatosensory cortex. Representative images at 6 months post-injection are shown in FIG. 6A.

Quantification of the GFAP-positive area 4 and 6 months after treatment shows that astrocyte activation was significantly reduced in both brain regions in the scAAV9.P546.CLN3-injected CLN3^{Δex7/8} mice compared to PBS-injected CLN3^{Δex7/8} mice (FIG. 6B). Although, the level of GFAP staining in scAAV9.P546.CLN3-injected CLN3^{Δex7/8} mice in these brain areas was much lower compared to PBS treated CLN3^{Δex7/8} mice, they remained above wild type levels at both 4 and 6 months post injection for most areas analyzed.

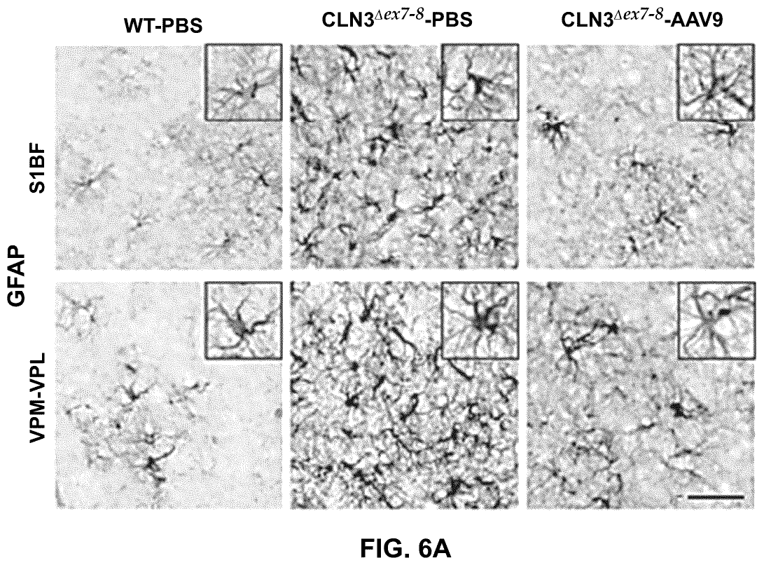

Glial activation was also determined in VPM/VPL and somatosensory cortex sections using anti-CD68 staining as a marker for activated microglia. CD68 is a lysosomal protein that is upregulated in cells primed for pro-inflammatory functions such as phagocytosis (Seehafer et al., J Neuroimmunol. 2011; 230:169-172). Similar to what was observed with astrocytes, glial activation was significantly reduced in the VPM/VPL and somatosensory cortex regions in the AAV9 injected CLN3^{Δex7/8} mice compared to PBS-injected CLN3^{Δex7/8} mice after 4 months (FIG. 7A-B). In the somatosensory cortex, treatment with scAAV9.P546.CLN3 reduced CD68 staining to a level that was comparable to wild type mice. At the 6 month time point, there was no significant improvement in the level of CD68 staining in scAAV9.P546.CLN3-treated compared to PBS-treated CLN3^{Δex7/8} mice in the VPM/VPL region, however, there was still a significant reduction in reactive glia in the somatosensory cortex of the scAAV9.P546.CLN3-treated mice (FIG. 7A-B).

Behavioral Improvements Following Delivery of scAAV9.P546.CLN3

In human CLN3-Batten disease patients, neurological deficits such as motor and cognitive dysfunction become apparent much later compared to earlier-onset disease variants such as CLN3-Batten disease (late-infantile Batten disease), which might be due to residual function of the truncated CLN3 protein (Kitzmüller et al., Hum Mol Genet. 2008 Jan. 15; 17(2):303-12). This delay in phenotype is also present in the CLN3^{Δex7/8} mouse model. In the efficacy study for scAAV9.P546.CLN3, starting at 2 months of age, and continuing at 2-month intervals, mice were subjected to a battery of behavioral testing paradigms including: accelerating rotarod assays and pole climbing to test motor function and coordination, as well as Morris water maze to assess learning and memory. Currently, animals have been followed for 10 months post-injection and studies are ongoing. Previous publications characterizing this mouse model indicate initial delay in neurodevelopmental behavior, followed by normalization and later decline starting at around 10-12 months of age (Osório et al., Genes Brain Behav. 2009 April; 8(3): 337-345).

Rotarod analysis showed no statistically significant differences between wild type and PBS or treated CLN3^{Δex7/8} mice up to 18 months post injection.

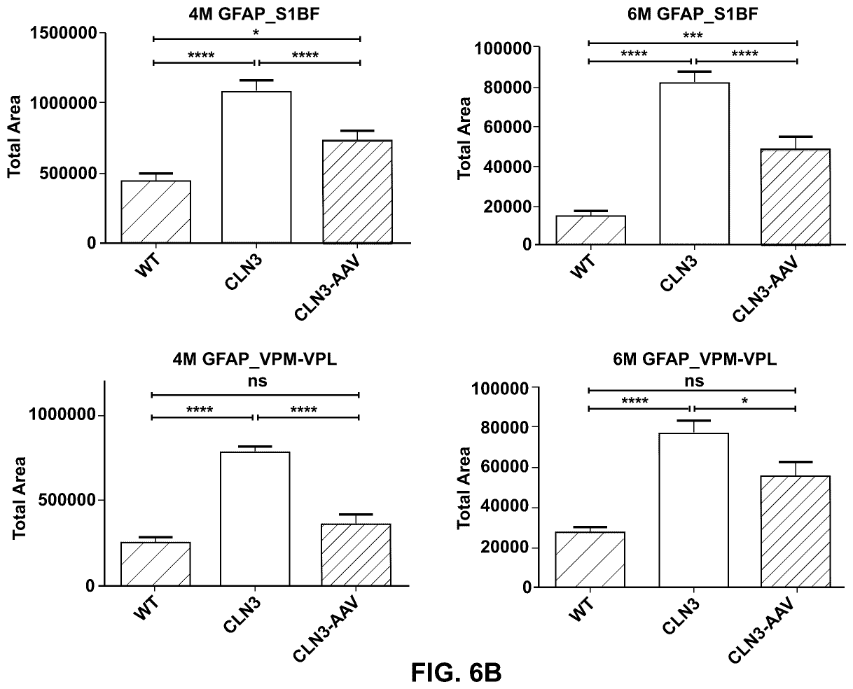

The rotarod assay was performed every 2 months. Mice were placed on an accelerating wheel and time until they fell was measured. At each time point, mice were trained in the morning and testing was performed 4 hours later in the afternoon. Unlike previously published data, no significant difference in performance of wild type mice and PBS CLN3^{Δex7/8} mice up to 18 months post injection was observed (Bosch et al., J Neurosci. 2016; 36(37): 9669-9682). However, a significant difference in latency to fall was observed at 2 months post injection in female WT mice versus PBS treated CLN3^{Δex7/8} mice. This discrepancy compared to previous data most likely lies in the design of the testing protocol and/or environmental factors in housing. The current protocol used in this study is testing the animals only at one day at each time point, whereas previously published data repeated testing over a time span of 4 days. Moreover, the protocol used in this study was performed at a slightly lower starting speed (36 rpm vs. 40 rpm) and with a longer time interval between morning training and afternoon testing period compared to previously published data (4 hours break vs. 2 hours break). Moreover, the am training set-up was also different: while in the previous study, the mice were trained on a wheel spinning at a constant 5 rpm only in the morning for 5 min, the animals in the current study were trained using the very same settings that were then applied in the afternoon testing, which leads to acceleration of the wheel by 0.3 rpm every 2 seconds. In summary, at up to 18 months post injection, no deficits in the ability to hold on to an accelerating rotarod wheel was observed in untreated or scAAV9.P546.CLN3 treated CLN3^{Δex7/8} mice compared to wild type animals (FIG. 8, top panel) with the described settings.

Morris water maze analysis showed statistically significant differences between wild type and CLN3^{Δex7/8} mice at 2, 4, 16 and 18 months post injection.

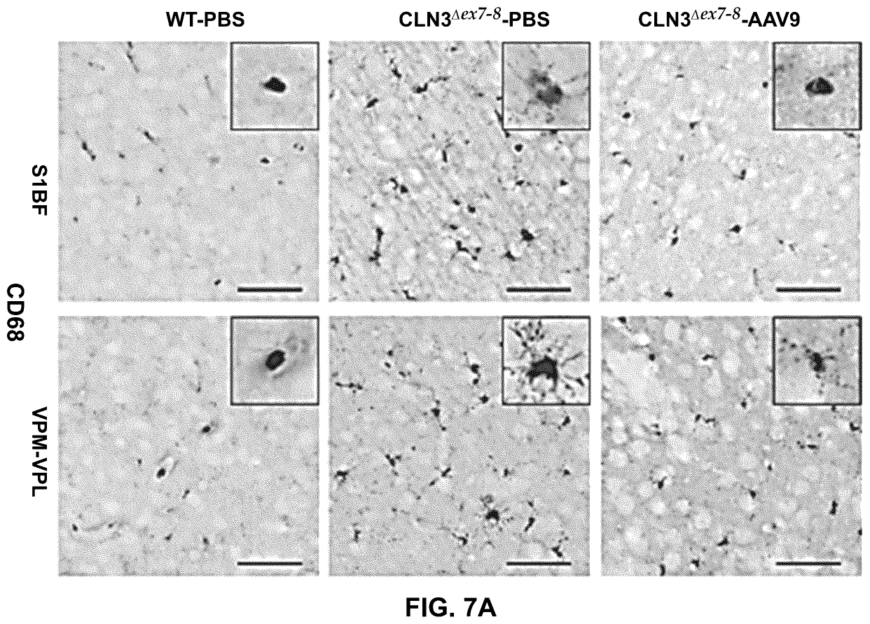

In the Morris Water Maze test, animals were placed in a water-filled pool containing a hidden platform. After training, the time it took the animals to find the hidden platform using environmental cues for orientation was measured as a sign of learning and memory capabilities. At 2 and 4 months post injection, statistical differences were observed between wild type animals and PBS or scAAV9.P546.CLN3 treated CLN3^{Δex7/8} mice, indicating that at this time point of the disease, the learning and memory was impaired to be measurable by this test resulting in a latency for the animals to find the hidden platform. Furthermore, more significant statistical differences in latency were observed between wild type and PBS or scAAV9.P546.CLN3 treated CLN3^{Δex7/8} mice at 16 and 18 months (FIG. 9, top left panel). The increased latency at 16 months also correlated with increased swim speed for the PBS treated CLN3^{Δex7/8} mice (FIG. 9, top right panel). In addition, when separated by gender, statistical differences in latency were observed between male wild type animals and scAAV9.P546.CLN3 treated CLN3^{Δex7/8} mice at 16 and 18 months (FIG. 9, middle left panel), while the scAAV9.P546.CLN3 treated CLN3^{Δex7/8} male mice swim speed significantly decreased at 16 months (FIG. 9, middle right panel). The scAAV9.P546.CLN3 treated female CLN3^{Δex7/8} mice showed significant increased latency compared to wildtype or PBS treated CLN3^{Δex7/8} animals at 18 months (FIG. 9, bottom left panel), while the PBS treated CLN3^{Δex7/8} male mice swim speed was significantly increased at 16 months (FIG. 9, bottom right panel).

Pole climbing assay showed improved performance of scAAV9.P546.CLN3 treated CLN3^{Δex7/8} compared to PBS injected animals.

The pole climbing test measures the time the mice take to turn around on a vertical pole when placed on it facing upwards, as well as the time to descend the pole when placed on it facing downwards. Moreover, the number of falls from the pole while attempting to turn or descend is also sometimes measured. This test evaluates coordination and balancing capabilities.

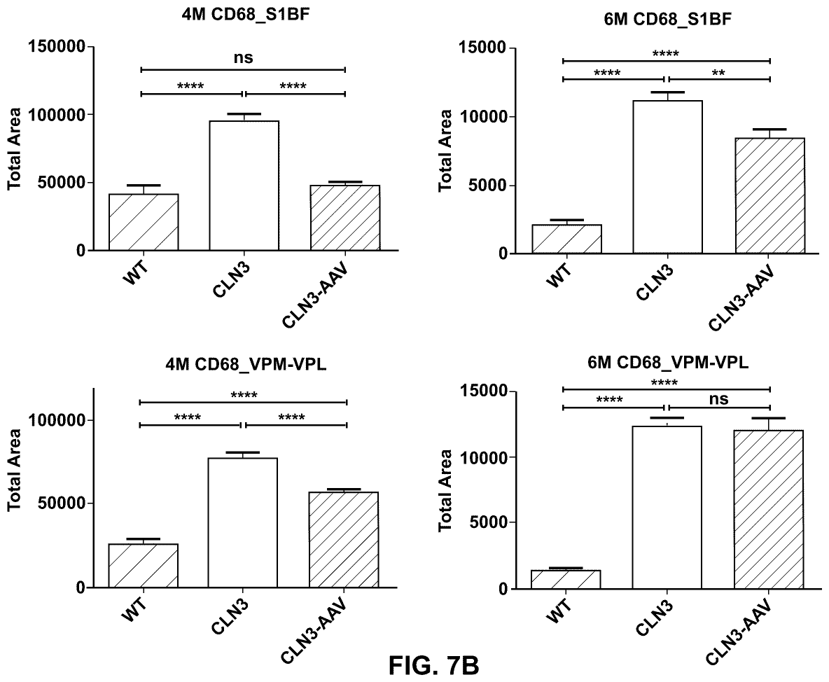

At 10 and 12 months post injection, scAAV9.P546.CLN3 animals were significantly faster in descending the pole compared to PBS treated animals (FIG. 10, top left panel). Statistically significant differences were seen in the time it took PBS treated CLN3^{Δex7/8} animals to descend a pole at 10 and 12 months post injection, while wild type and scAAV9.P546.CLN3 treated CLN3^{Δex7/8} were indistinguishable (FIG. 10, top left panel). Regarding the time it took the animals to turn from a facing upwards position to facing downwards, two statistically significant differences were found. At 2 and 16 months of age, wild type animals turned significantly faster compared to both scAAV9.P546.CLN3 and PBS treated CLN3^{Δex7/8} mice. This difference was more pronounced in male mice (FIG. 10, middle left panel) compared to females (FIG. 10, bottom left panel) where no difference compared to wild type was seen. The 2 and 16 month time points were the only time points in which differences in this parameter were observed between study groups (FIG. 10, top left panel).

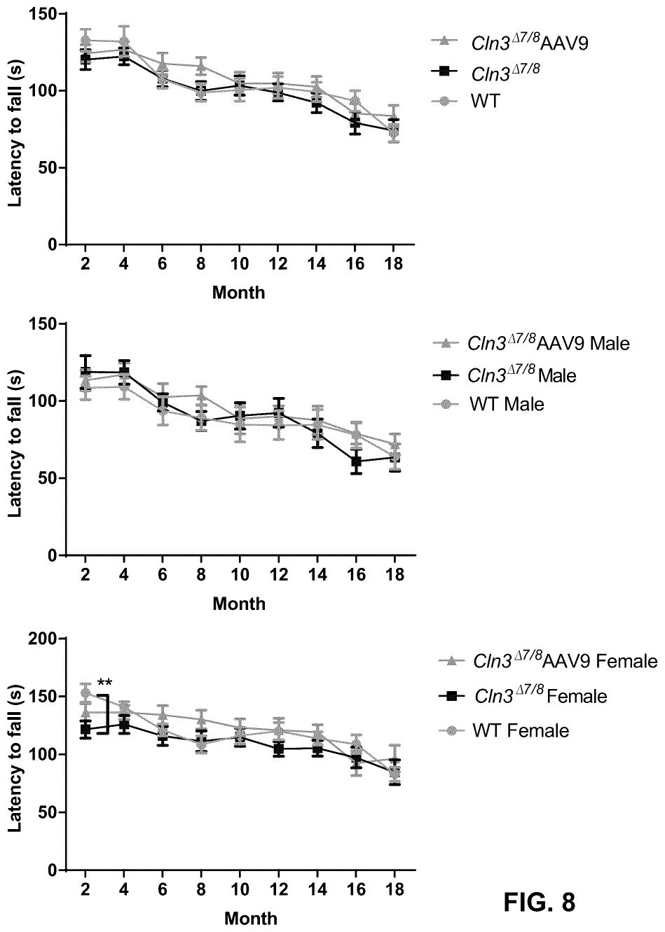

Additional statistically significant differences were seen in the average number of falls from the pole, where PBS treated CLN3^{Δex7/8} males and females fell off more frequently compared to wild type and scAAV9.P546.CLN3 treated animals (FIG. 11). Top graph: Significant differences were found in month 2 in the number of falls between scAAV9.P546.CLN3 treated animals and PBS treated animals. A statistically significant difference was also observed between the wildtype and PBS treated CLN3^{Δex7/8} mice at 16 months post injection. Middle graph: males only. PBS treated CLN3^{Δex7/8} mice fell more often from the pole than other treatment groups, with the greatest statistical significance at 16 months post injection. Bottom graph: Differences in falls were significant at 8 months post injection for females, but the trend was visible during the whole study. N=5 for each treatment group (5M/5F). Interestingly, differences in falls from the pole were seen during the whole 18 months and were statistically significant at an early time point (4 months) as well as at 8 months and 16 months. At 8 months, the difference was statistically significant only in females, but a clear trend existed also in males and was statistically significant in the males at 16 months post injection. In general, PBS treated CLN3^{Δex7/8} males were falling off the pole more frequently than other treatment groups.

In summary, there is strong evidence that treatment of CLN3^{Δex7/8} mice with scAAV9.P546.CLN3 prevents the accumulation of ASM material, as well as ATP synthase subunit C, both major hallmarks of CLN3-Batten disease progression. These data correlate with a strong reduction in glial activation (astrocytes and microglia). Although early in the disease course, first trends towards improvement of behavioral phenotypes are becoming evident: scAAV9.P546.CLN3 treated CLN3^{Δex7/8} mice were more capable of descending a vertical pole compared to PBS treated animals as they moved faster and fell off less often. Altogether, these data support scAAV9.P546.CLN3 gene therapy as a therapeutic strategy for this disease.

### Example 3

### Expression Studies With scAAV9.P546.GFP in Mice

The P546 promoter allows expression of transgenes throughout the CNS in a similar manner as the chicken-beta-actin (CBA) promoter. To do a side-by-side comparison between the two promoters, at post-natal day 1, mice were injected with either scAAV9.CB.GFP or scAAV9.P546.GFP formulated in 1× PBS and 0.001% Polyoxyethylene-polyoxypropylene block copolymer (PLURIONIC® F-68) or 20 mM Tris (pH8.0), 1 mM MgCl2, 200 mM NaCl, 0.001%. poloxamer 188 at 5×10¹⁰ viral genomes per animal. After 3 weeks, the animals were sacrificed and the brains were put directly under a fluorescent dissection microscope. From the fluorescent images, it was evident that the GFP distribution was similar, but the level of fluorescence was lower in the animal that received the scAAV9.P546.GFP compared to the one that received scAAV9.CB.GFP, confirming that the P546 promoter led to a more moderate expression level of the transgene compared to the CBA promoter.

Another mouse was injected with scAAV9.P546.GFP and kept alive for 200 days. After 200 days, the animal was sacrificed and whole-brain saggital sections were stained for GFP expression. Even 200 days post injection, widespread expression of the GFP transgene was observed throughout the entire brain including Cortex, Hippocampus, Midbrain, Medulla, Amygdala and Cerebellum, further indicating that the P546 promoter is a good candidate for CNS gene therapy.

The data from GFP fluorescence and GFP immunofluorescence staining was further supported by western blot data from various tissues and brain areas. GFP expression was readily detectable three weeks post injection with fluorescent western blot technique using an imaging system (such as the LI-COR® System) in mice that were treated with scAAV9.P546.GFP (n=3) while no band was detected in a PBS injected animal that was used as control (n=1). Transgene expression was evident in whole brain lysates, as well as region specific lysates including cortex, medulla, midbrain, hippocampus, cerebellum and spinal cord.

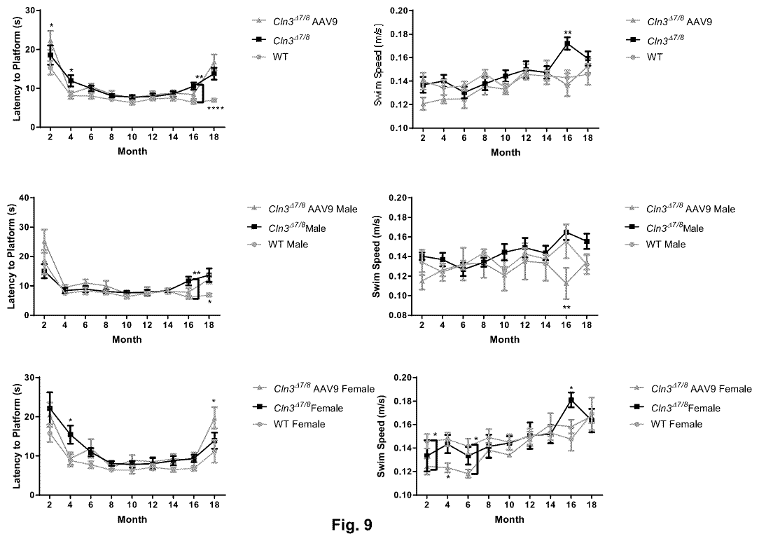

Moreover, GFP expression was also confirmed in heart and liver, while lung and spleen showed little to no transcript expression (FIG. 12). The western blot data with scAAV9.P546.GFP is consistent with the expression data from the mouse and nonhuman primate safety studies, where a very similar expression profile was found. Moreover, this expression pattern in brain and peripheral organs is comparable to the pattern found with scAAV9.CB.GFP.

In summary, extensive expression analysis in mice using immunostaining and western blot techniques indicates that the P546 promoter leads to a very similar and long-lasting expression profile throughout the nervous system while allowing a more moderate expression level compared to the strong CBA promoter.

### Example 4

### Expression Studies With scAAV9.P546.CLN3 in Non-Human Primates

A single dose of 3.4×10¹³ vg scAAV9.P546.CLN3 was in 1× PBS and 0.001% Polyoxyethylene-polyoxypropylene block copolymer (PLURIONIC® F-68) and administered into three 3-4 year old cynomolgus macaques.

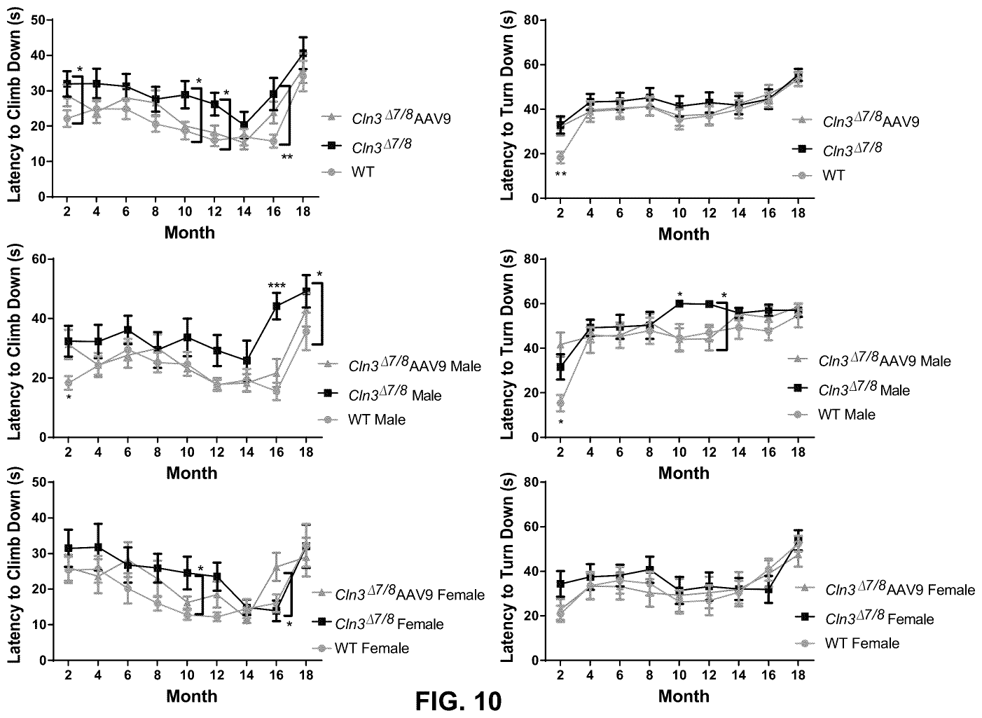

For the targeting analysis in brain tissue of the cynomolgus macaques that were injected with scAAV9.P546.CLN3, the targeting was analyzed on the RNA level using primers that are specific for the human CLN3 transgene and do not cross-react with the endogenous non-human primate CLN3 RNA. Reverse transcription quantitative PCR in tissue from various brain regions of one of the cynomolgus macaques that was sacrificed 12 weeks post injection revealed expression of human CLN3 in all levels of the spinal cord, cortex, thalamus, striatum, cerebellum and retina, further underlining the widespread reach of scAAV9 and the expression of the transcript throughout the brain and spinal cord with the P546 promoter (FIG. 13). Of note, the primers used for the detection of vector-derived human CLN3 do not cross-react with the endogenous NHP CLN3 transcript. Thus, the normalization was performed against the vector-derived CLN3 RNA levels found in the lumbar spinal cord which was set to 1 rather than to saline-injected or uninjected animals as a normalization to zero was not possible. Actin was used as a normalizing gene.

In summary, the data from non-human primates prove the high potential of scAAV9 to travel through the nervous system and reach large regions of the CNS after a single intrathecal lumbar injection. Of note, all non-human primates treated via intrathecal injection of scAAV9.P546.CLN3 tolerated the treatment well and no adverse effects were observed in any of the animals at any time point up to 6 months post injection.

### Example 5

### Clinical Trial of scAAV9.P546.CLN3 Gene Therapy

The scAAV9.P546.CLN3 will be delivered intrathecally to human patients with CLN3-Batten Disease.

The scAAV for the clinical trial is produced by the Nationwide Children's Hospital Clinical Manufacturing Facility utilizing a triple-transfection method of HEK293 cells, under cGMP conditions as described in Example 1.

Patients selected for participation will be 3-10 years of age with a diagnosis of CLN3 disease as determined by genotype. The first cohort (n=3) will receive a one-time gene transfer dose of 6×10¹³ vg total scAAV per patient. The scAAV9.P546.CLN3 is formulated in 20 mM 1 mM MgCl₂, 200 mM NaCl, 0.001%. poloxamer 188 Tris (pH 8.0), and will be delivered one-time through an intrathecal catheter inserted by a lumbar puncture into the interspinous into the subarachnoid space of the lumbar thecal sac. Safety will be assessed on clinical grounds, and by examination of safety labels. There will be a minimum of four weeks between enrollments of each subject to allow for review of Day 30 post-gene transfer safety data. If there are no safety concerns, after the third subject is evaluated at one month post-injection, a second cohort of four additional subjects will be enrolled. Each subject in cohort 2 (n=4) will receive an escalated dose of 1.2×10¹⁴ vg total scAAV. There will be at least a six week window between the completion of Cohort 1 and the start of Cohort 2, to allow review of the safety analysis from five time points (days 1, 2, 7, 14, and 21) as well as DSMB review prior to dosing of the next subject.

Disease progression will be measured with the UBDRS scales (referenced in the Detailed Description above) and the impact of treatment on quality of life using the Pediatric Quality of Life (PEDSQOL) scale, and potential for prolonged survival.

The primary analysis for efficacy will be assessed when all patients have completed the three-year study. Basis of determining efficacy will be by stabilization or reduced progression of the disease based on the well-established Unified Batten Disease Rating Scale (UBDRS) that was developed specifically for CLN3-Batten Disease. Upon completion of the three-year study period patients will be monitored annually for 5 years as per FDA guidance.

### Example 6

### Additional Studies in Cln3^{Δ7/8} Mice Model

As described in Example, 2 Wild type (WT) and Cln3Δ7/8 mice were dosed with either PBS, scAAV9.p546.CLN3, or scAAV9.CB.CLN3 gene therapy via intracerebroventricular (ICV) injection at postnatal day 1. In this study, the mice were administered 5×1010 vg/animal (4 μL volume).

The injection method and timing was selected to target specific neuronal populations that are relevant in CLN3-Batten disease patients. Animals were sedated via hypothermia during the procedure, monitored until fully recovered, and genotyped as previously described (see Morgan et al. PLOS One 8; and Laboratory, TJ Protocol 18257: Standard PCR Assay).

Statistical analyses were performed using scientific graphing and statistics software (GraphPad Prism) and details are noted in the figure legends. In general, two-way ANOVA was employed with appropriate post-hoc test, and outliers were removed with the ROUT method, Q=0.1-1%. If appropriate, an unpaired t-test was used.

Expression and Distribution of hCLN3 Transcript in the Brain

Quantitative PCR was carried out to measure hCLN3 transcript in the brains of the treated mice. Total RNA and cDNA was generated as described previously (see Cain et al. Mol Ther., 2019). The 2\hat{ }-Delta-Delta Ct method was used to calculate relative gene expression of the human CLN3 transcript normalized to Gapdh as the housekeeping control. hCLN3 Forward primer sequence: CGCTAGCATCTCATCAGGCCTTG (SEQ ID NO: 11); hCLN3 Reverse primer sequence: AGCATGGACAGCAGGGTCTG (SEQ ID NO: 12).

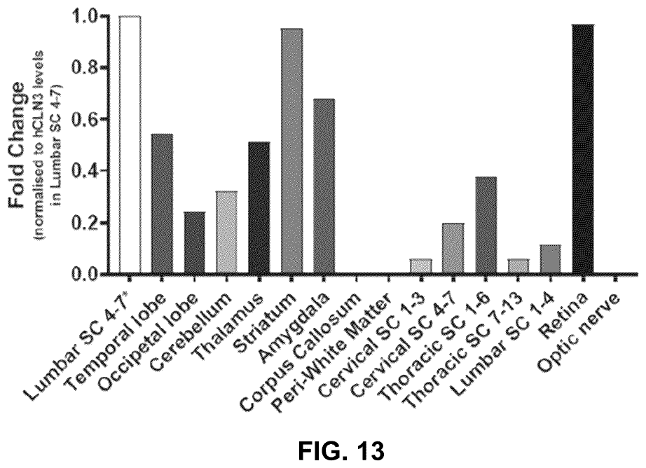

As shown in FIG. 16, scAAV9.p546.CLN3 treatment resulted in increased levels of hCLN3 transcript expression in the cerebral cortex and spinal cord of Cln3^{Δ7/8} mice as measured by qPCR up to 24 months of age. Thus, a single, neonatal, ICV administration of scAAV9.p546.CLN3 results in sustained and well-targeted expression of hCLN3.

In addition, in situ hybridization (RNASCOPE®) was carried out to detect CLN3 transcript in the brain of the treated mice. Mice were CO₂ euthanized and cardiac perfused with PBS. Brains were collected and placed on a 1 mm sagittal brain block. Brains were sliced at the midline and 3 mm right of the midline. The 3 mm sagittal piece was flash frozen with −50° C. isopentane and then sectioned on a cryostat at 16 μm and placed on slides. Slides were then processed according to the manufacturer suggested protocols (ACDBio manuals 320293 and 320513). Sections were labeled with a human specific CLN3 probe (ACDBio Cat No 470241), which consisted of 20 double Z pairs in regions of the CLN3 gene with little homology between mouse and human CLN3 (region 631-1711). Slides were fluorescently labeled with an in situ hybridization kit (RNASCOPE® Fluorescent Multiplex Kit, ACDBio Catalog no 320850) using their Amp 4-FL-AltC which tagged the hCLN3 probe with a 550 nm fluorophore, slides were counterstained with DAPI to label nuclei. Tissue sections were mounted on slides under coverslips using antifade mounting media (Dako faramount, Agilent). Slides were stored in the dark before imaging. Sections were imaged and analyzed using a microscope (Nikon NiE microscope with NIS-Elements Advanced Research software (v4.20)).

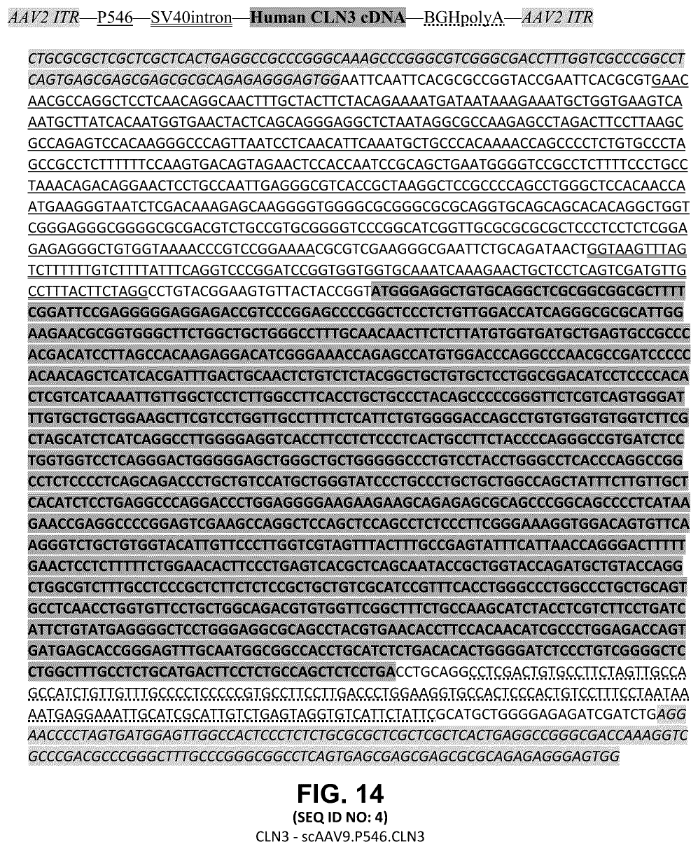

As shown in FIG. 17, scAAV9.p546.CLN3 treatment produces stable hCLN3 transcript throughout the brain of Cln3^{Δ7/8} mice as measured by in situ hybridization (RNASCOPE®) (red fluorescence), up to 24 months of age. The quantitative PCT and the in situ hybridization (RNASCOPE®) assays confirm a single, neonatal, ICV administration of scAAV9.p546. results in sustained and well-targeted expression of hCLN3. scAAV9.p546.CLN3 gene therapy increases hCLN3 gene expression throughout the brain and spinal cord up to 24 months of age.

### Classic Batten Disease Pathologies

To determine if administration of scAAV9.p546.CLN3 prevented classic Batten disease pathologies in the brains of Cln3^{Δ7/8} mice, storage material accumulation (ASM) and glial reactivity were examined after ICV administration. Wild type and Cln3^{Δ7/8} mice were CO₂ euthanized, perfused with PBS, and tissue was fixed with 4% PFA. Fixed brains were sectioned on a vibratome at 50 μm (Leica VT10008). Sections were processed with standard immunofluorescence and DAB staining protocols. Primary antibodies included anti-CD68 (AbD Serotec, MCA1957; 1:2000), anti-GFAP (Dako, Z0334; 1:8000), and anti-ATP synthase subunit C (Abcam, ab181243, 1:1000). Secondary antibodies included anti-rat and anti-rabbit biotinylated (Vector Labs, BA-9400; 1:2000). Sections were imaged and analyzed using a Slide Scanning Microscope at 20× (APERIO®). Images were extracted from the VPM/VPL of the thalamus and layers 2/3 of the somatosensory cortex, with multiple images taken of multiple tissues from each animal. Total area of immunoreactivity was quantified using a threshold analysis in ImageJ.

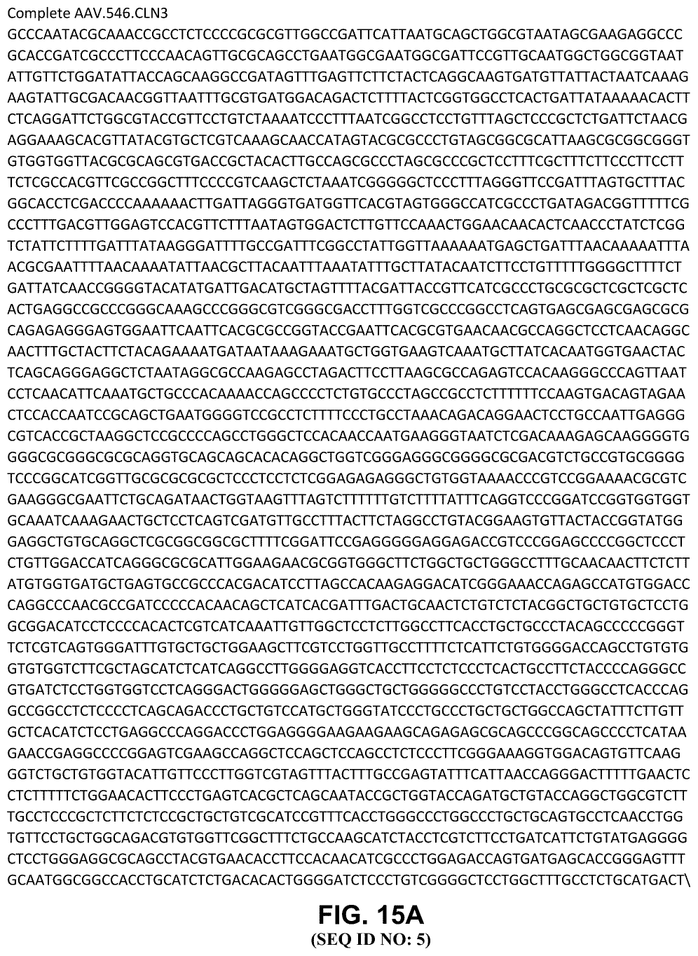

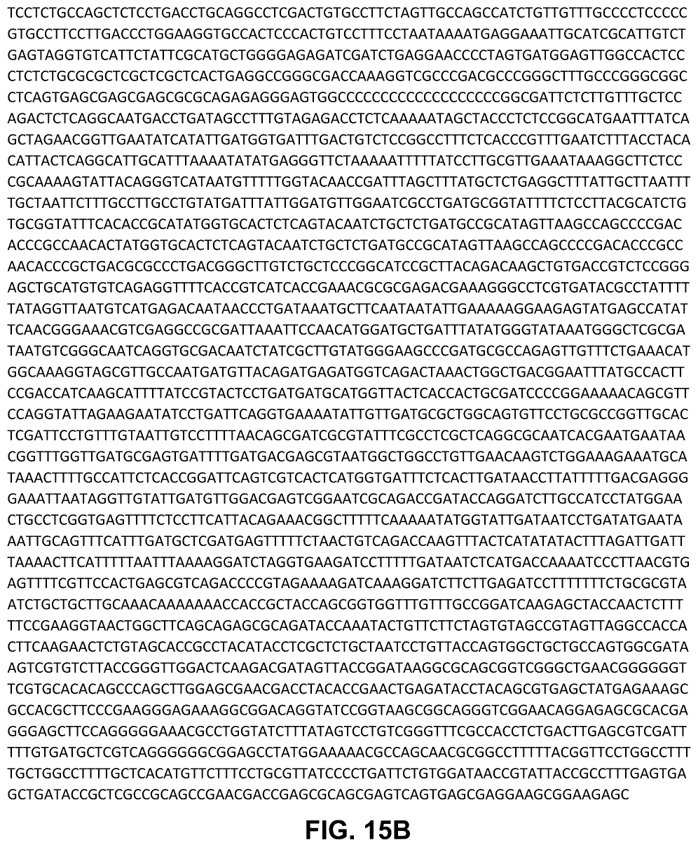

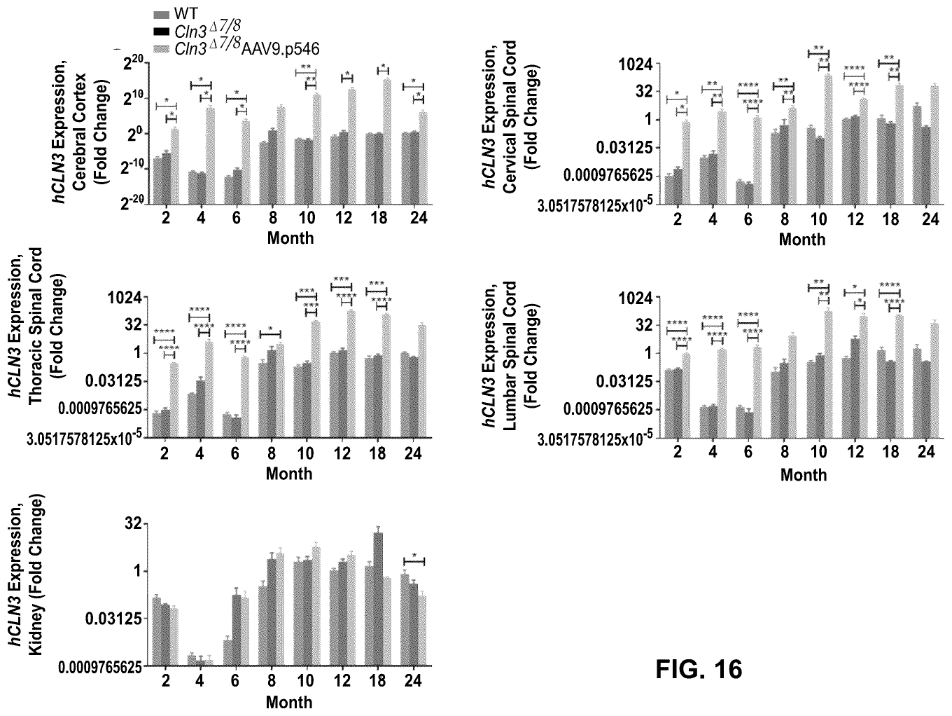

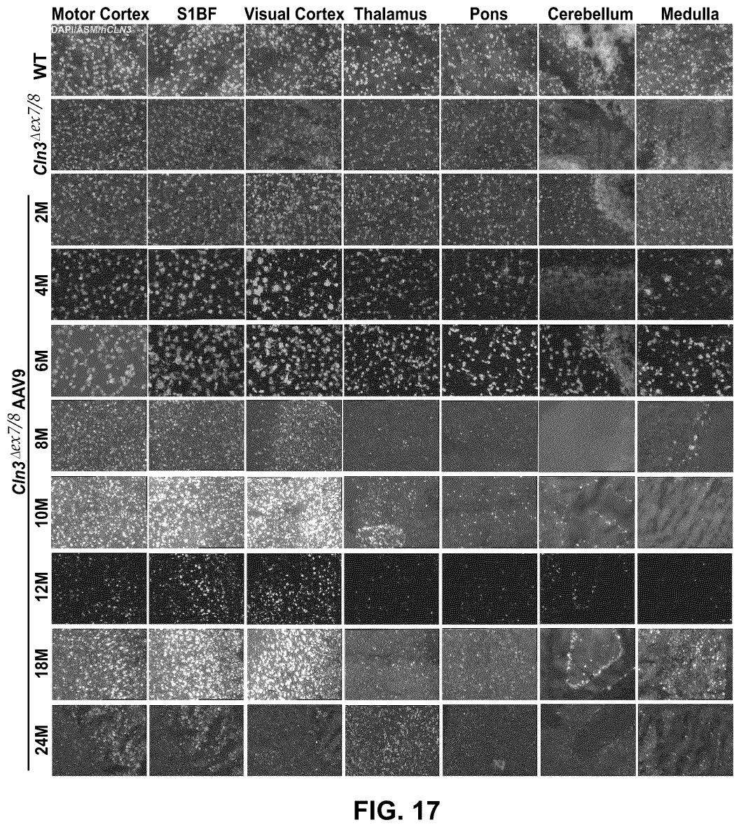

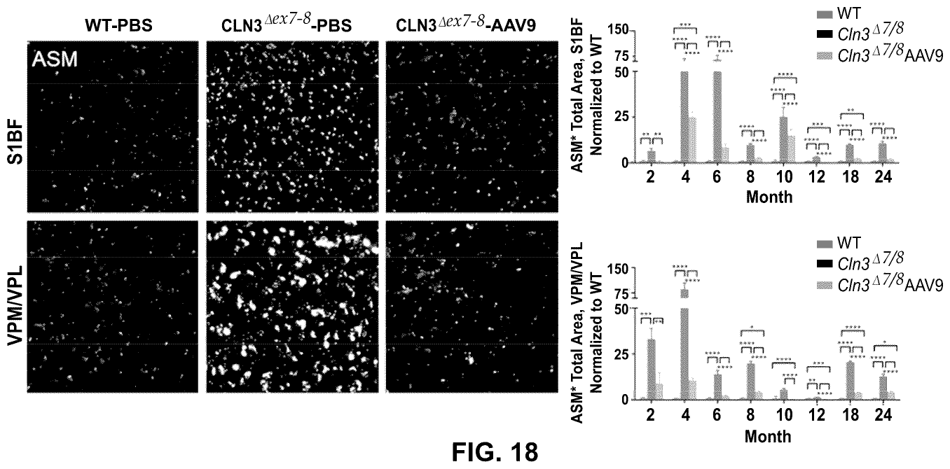

FIG. 18 demonstrates that scAAV9.p546.CLN3 treatment prevents and reduces ASM accumulation in two areas of the brain in Cln3^{Δ7/8} mice up to 24 months of age. FIG. 19 demonstrates that scAAV9.p546.CLN3 treatment generally prevented large amounts of SubUnitC accumulation, (a constituent of the ASM) in two areas of the brain of Cln3^{Δ7/8} mice up to 24 months of age. FIG. 20 demonstrates that scAAV9.p546.CLN3 treatment generally prevents astrocyte activation (GFAP+) in two areas of the Cln3Δ7/8 brain up to 24 months of age. FIG. 21 demonstrates that scAAV9.p546.CLN3 treatment prevents some microglial activation (CD68+) in the two areas of the Cln3Δ7/8 brain up to 24 months of age, dependent on time point. Thus, scAAV9.p546.CLN3 prevented classic Batten disease pathologies in the brain of Cln3^{Δ7/8} mice., including storage material accumulation and glial reactivity. FIG. 22 demonstrates that scAAV9.CB.CLN3 treatment is similarly effective in preventing various Batten disease pathologies in 6 and 12 month old Cln3^{Δ7/8} mice.

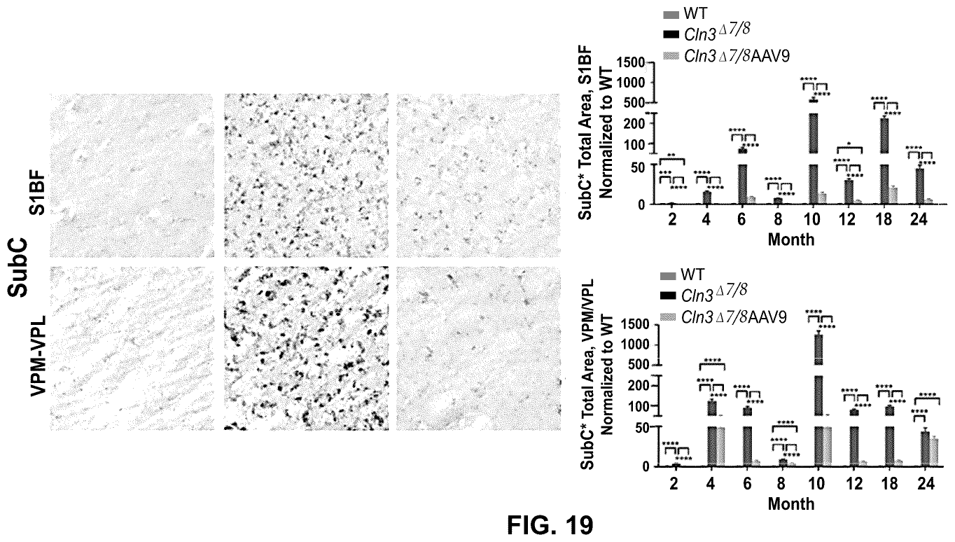

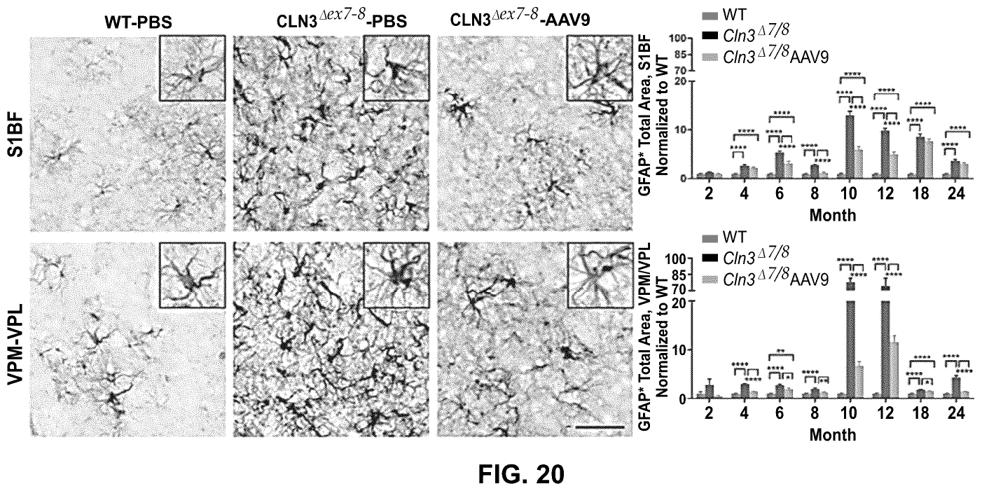

In addition, treatment with scAAV9.p546.CLN3 did not cause any red blood cells (CBC) abnormalities or while blood cell (WBC) abnormalities, as measured up to 24 months post-ICV administration. FIG. 23 provides data for the following CBCs parameters: RBC count, hemoglobin, hematocrit, mean corpuscular volume, mean corpuscular hemoglobin, mean corpuscular hemoglobin concentration, RBC distribution, platelet count and mean platelet volume. FIG. 24 provides data for the following WBCs parameters: WBC count, percent lymphocyte count, percent monocytes, percent granulocytes.

scAAV9.p546.CLN3 gene therapy prevents many of the cellular hallmarks of CLN3-Batten disease up to 24 months of age in Cln3^{Δ7/8} mice, including ASM, SubUnit C, GFAP and CD68 expression. In addition, scAAV9.CB.CLN3 gene therapy prevents many of the cellular hallmarks of CLN3-Batten disease up to 24 months of age in Cln3^{Δ7/8} mice, including ASM, SubUnit C, GFAP and CD68 expression.

### Example 7

### Sex-Based Histopathology Analysis in Cln3^{Δ7/8} Mice Model

As described in Example 2, Wild type (WT) and Cln3Δ7/8 mice were dosed with either PBS, scAAV9.p546.CLN3, or scAAV9.CB.CLN3 gene therapy via intracerebroventricular (ICV) injection at postnatal day 1. In this study, the mice were administered 5×10¹⁰ vg/animal (4 μL volume).

Wild type and Cln3^{Δ7/8} mice were CO₂ euthanized, perfused with PBS, and tissue was fixed with 4% PFA. Fixed brains were sectioned on a vibratome at 50 μm (Leica VT10008). Sections were processed with standard immunofluorescence and DAB staining protocols. Primary antibodies included anti-CD68 (AbD Serotec, MCA1957; 1:2000) and anti-ATP synthase subunit C (Abcam, ab181243, 1:1000). Secondary antibodies included anti-rat and anti-rabbit biotinylated (Vector Labs, BA-9400; 1:2000). Sections were imaged and analyzed using an Slide Scanning Microscope at 20× (APERIO®). Images were extracted from the following regions: CA2/CA3 region of the hippocampus, polymorphic layer of the hippocampal dentate gyrus, basolateral amygdala, habenula, reticular nucleus of the thalamus, ventral posterolateral/ventral posteromedial nucleus of the thalamus, mediodorsal and submedial regions of the thalamus, piriform cortex, retrosplenial cortex, and layers 2/3 of the somatosensory cortex, with multiple images taken of multiple tissues from each animal. Total area of immunoreactivity was quantified using a threshold analysis in ImageJ.

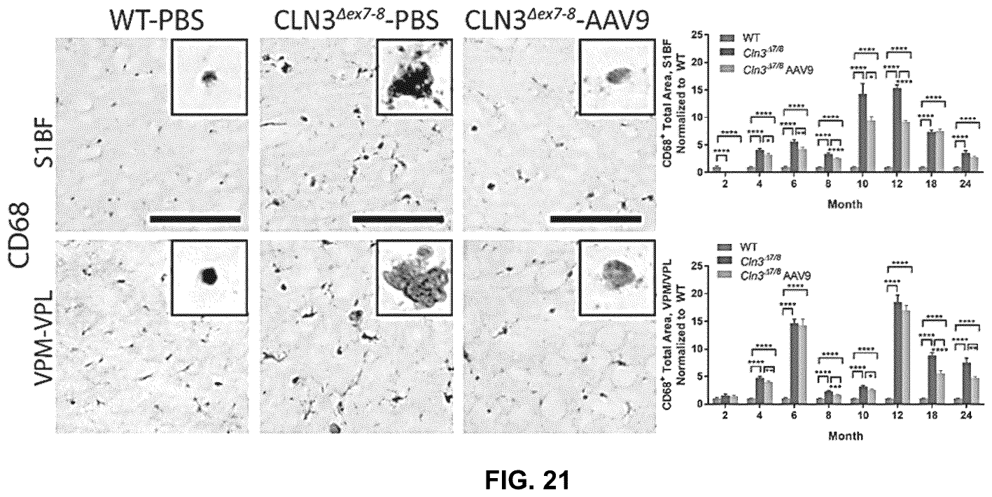

FIG. 25 demonstrates that mice treated with scAAV9.p546.CLN3 show differing levels of SubC accumulation in the CA3 region of the hippocampus based on sex at 12 months of age. At 12 months of age, treated female Cln3^{Δ7/8} mice accumulated significantly more SubC than wild type, while the accumulation of SubC in treated males did not differ from wild type. However, this difference was not seen at any other time point analyzed.

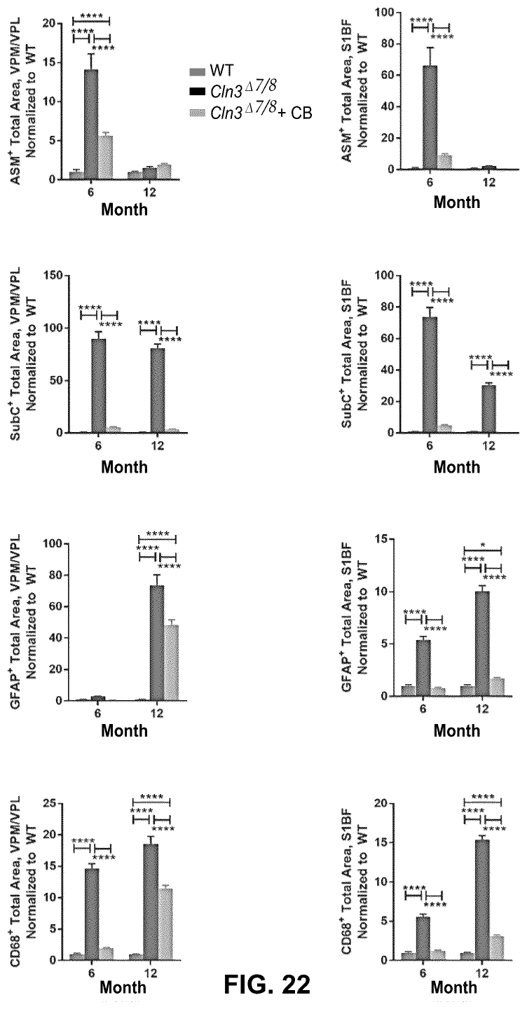

FIG. 26 demonstrates mice treated with scAAV9.p546.CLN3 showed subtle, differing levels of SubC accumulation in the Piriform Cortex (PIRC) based on sex at multiple time points. At 12 months of age, treated female mutant CLN3 mice accumulated significantly more SubC than wild type with AAV treatment not preventing accumulation below PBS mutant levels, while the accumulation of SubC in treated males did not differ from wild type. However, this correlation was not seen at any other time point analyzed and was inconsistent with findings at 18 months of age where SubC accumulation in treated females was not significantly different than wild type and was significantly lower than untreated mutant mice. At 18 months of age, accumulation of SubC in treated males becomes significantly higher than WT levels.

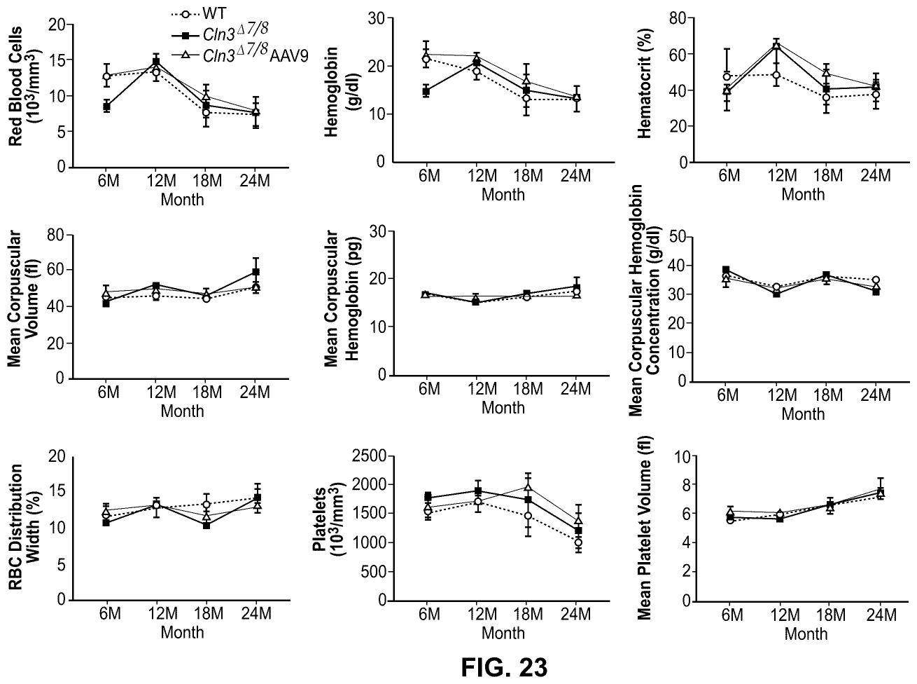

FIG. 27 demonstrates scAAV9.p546.CLN3 treated mice showed differing levels of SubC accumulation in the Reticular Thalamic Nucleus (RTN) based on sex at multiple time points. At 6 months of age, treated female mutant CLN3 mice accumulated significantly more SubC than wild type, while the accumulation of SubC in treated males does not differ from wild type. At 12 months of age, treated males remained at wild type levels, while treated females have significantly more SubC than both wild type and untreated mutant mice. This difference between female treated and untreated mutants was not present at 18 months, where SubC was significantly higher than WT but significantly lower than untreated mutants in both males and females.

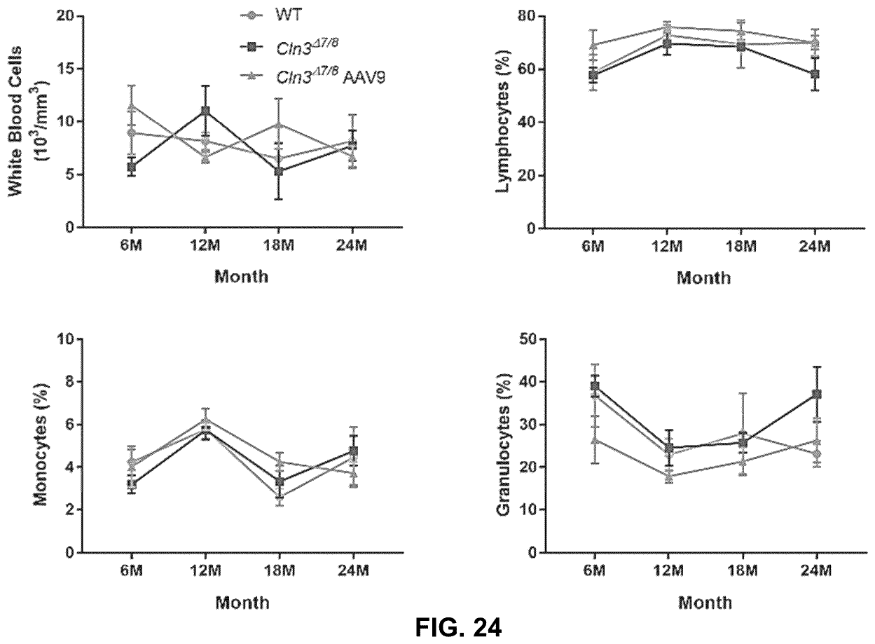

FIG. 28 demonstrates that scAAV9.p546.CLN3 treated mice show differing levels of SubC accumulation in the Somatosensory Cortex based on sex at 12 months. At 12 months of age, treatment with AAV did not reduce SubC accumulation compared to untreated mutant females, while accumulation of SubC in treated males was prevented and did not differ from wild type. However, this difference was not seen at any other time point analyzed.

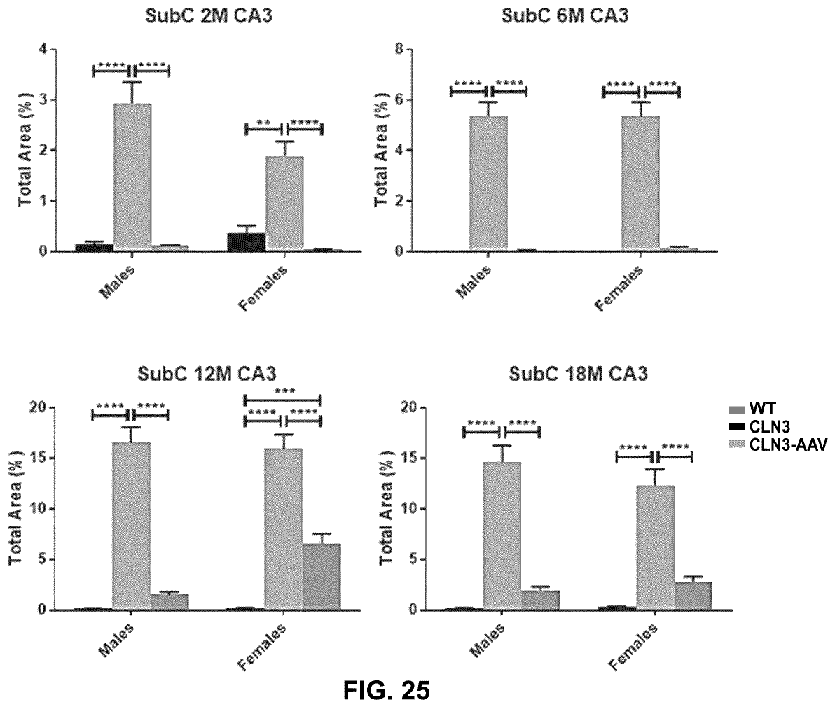

FIG. 29 demonstrates mice treated with scAAV9.p546.CLN3 showed differing levels of SubC accumulation in the VPM/VPL of the thalamus based on sex at 12 months. At 6 months of age, treated females accumulated significantly less SubC than untreated mutant females, but significantly more than wild type females. This difference continued through 12 and 18 months, with no differences between wild type and treated males at any time point analyzed.

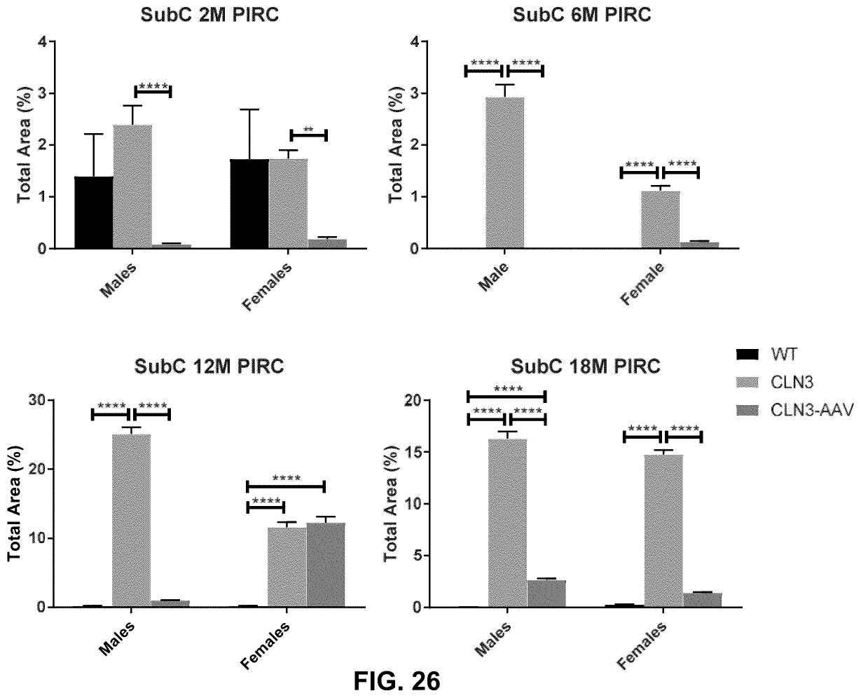

FIG. 30 demonstrates mice treated with scAAV9.p546.CLN3 show differing levels of SubC accumulation in the Basolateral Amygdala (BLA) based on sex at 12 months. At 12 months of age, AAV did not significantly prevent accumulation of SubC in treated female animals. At 18 months, SubC accumulation in treated females remained significantly higher than wild type, but was lower than untreated females. By 18 months treated males also begin to have significantly more SubC than their wild type counterpart, mimicking what was seen in female groups.

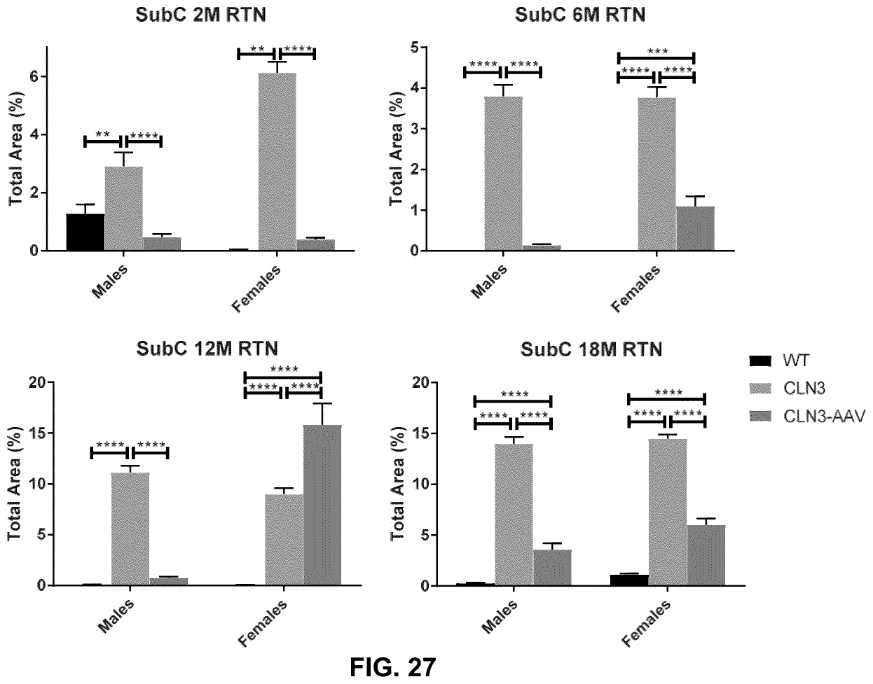

FIG. 31 demonstrates scAAV9.p546.CLN3 treated mice showed differing levels of SubC accumulation in the Polymorphic Layer of the Dentate Gyrus (DG) based on sex at 12 and 18 months. At 12 months of age, treated females appeared to have accumulated significantly more SubC than both wild type females and untreated mutant females. Raw images (not shown) revealed a darkened granule cell layer surrounding the polymorphic layer of the dentate gyrus that may impact the thresholding results. This darkening was present only in this group and only at the 12 month time point. This increase was also no longer seen at 18 months in females, however, at 18 months treated males began to have more SubC accumulation than wild type males.

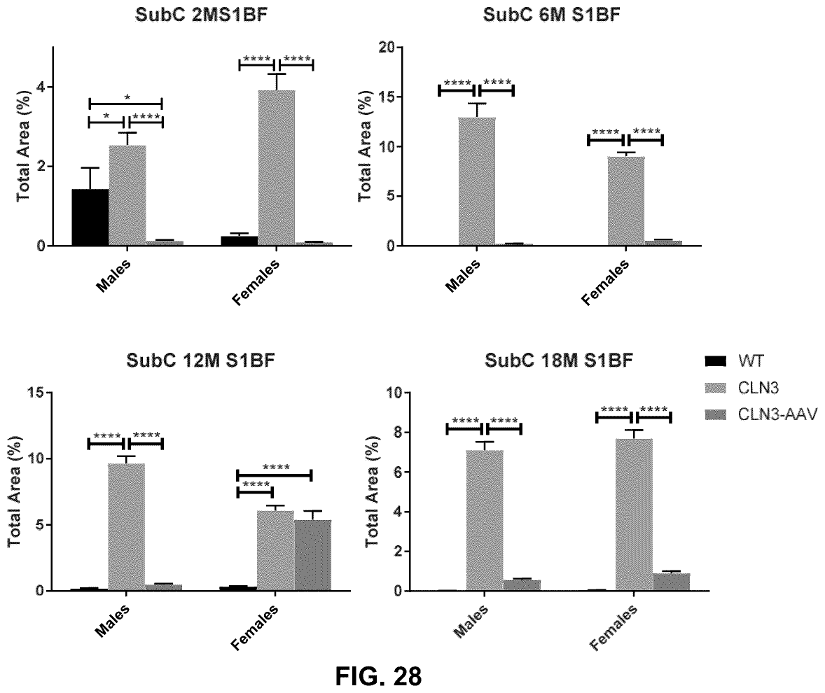

FIG. 32 demonstrates scAAV9.p546.CLN3 treated mice show differing levels of SubC accumulation in the Habenula based on sex at 12 and 18 months. At 12 months of age, treated females accumulated significantly less SubC than untreated mutant females, but significantly more than wild type females. This difference was also seen at 18 months, with no differences between wild type and treated males at any time point analyzed.

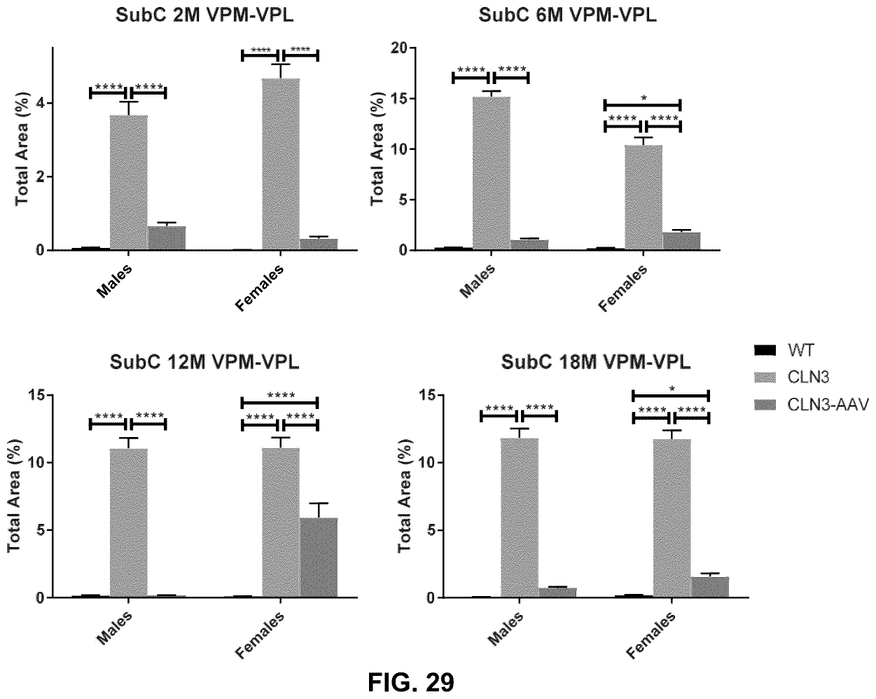

FIG. 33 demonstrates mice treated with scAAV9.p546.CLN3 showed differing levels of SubC accumulation in the Mediodorsal Nucleus based on sex at 12 and 18 months. At 12 months of age, treated females accumulated significantly less SubC than untreated mutant females, but significantly more than wild type females. This difference was also seen at 18 months, with no differences between wild type and treated males at any time point analyzed.

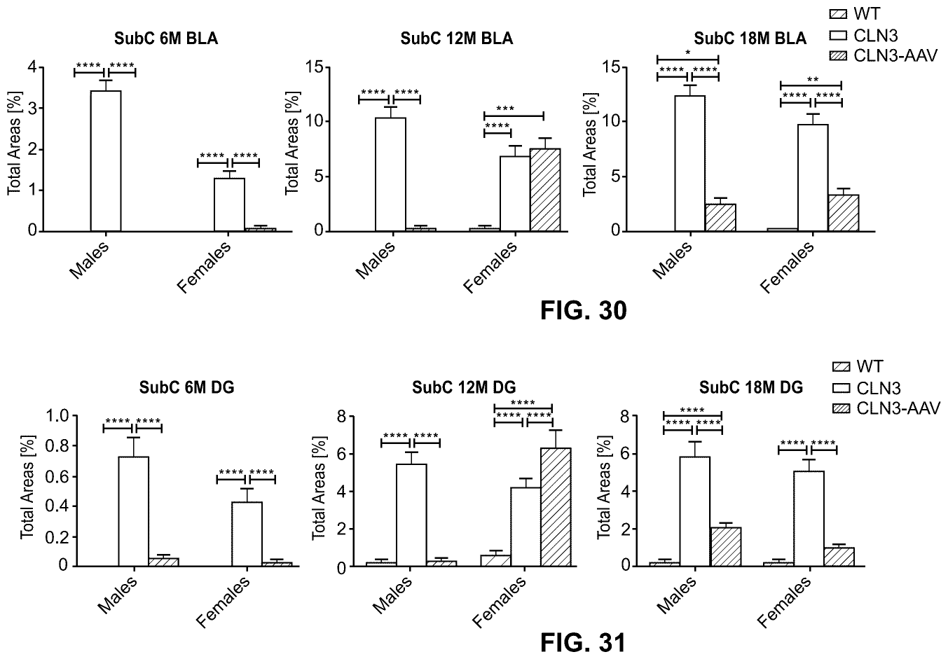

FIG. 34 demonstrates mice treated with scAAV9.p546.CLN3 showed no difference in levels of SubC accumulation in the Retrosplenial Cortex (RSC) based on sex. There were no differences between wild type and treated males at any time point analyzed.

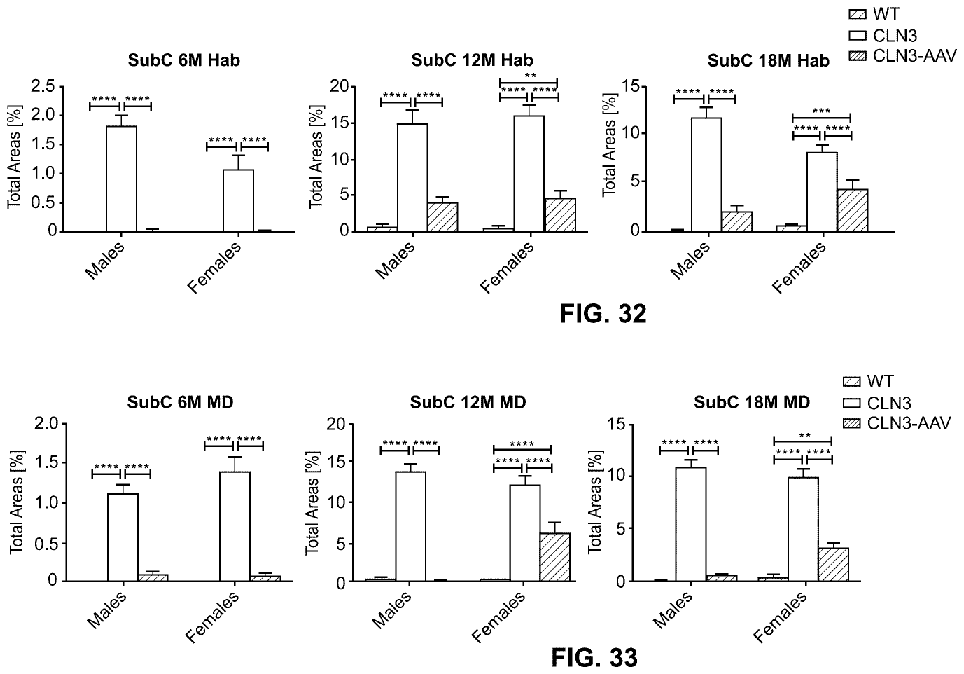

FIG. 35 demonstrates mice treated with scAAV9.p546.CLN3 showed differing levels of activated microglia (CD68⁺) in the Somatosensory Cortex (SIBF) based on sex at 6 months. At 6 months of age, treated females had increased microglial activation compared to wild type and untreated females, with treated males higher than wild type but lower than untreated. No sex differences were seen at 12 and 18 months of age.

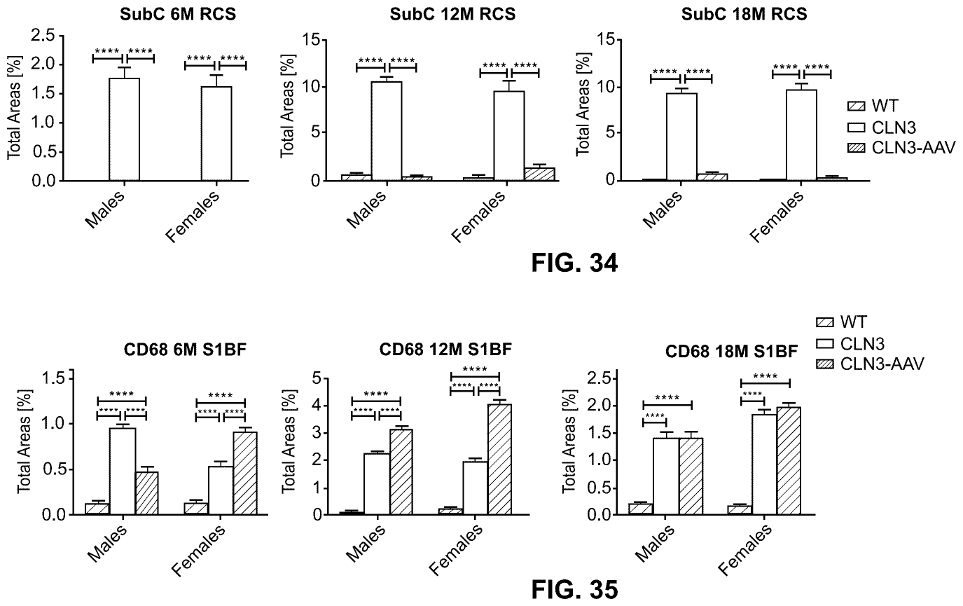

FIG. 36 demonstrates mice treated with scAAV9.p546.CLN3 show differing levels of microglia activation in the VPM-VPL, thalamus based on sex. At 6 months of age, treated females have increased microglial activation compared to wild type and untreated females, with treated males higher than wild type but lower than untreated. This difference is also seen at 12 months. The same is seen in female groups at 18 months, however, treated males did not significantly differ from wild type at this time point.

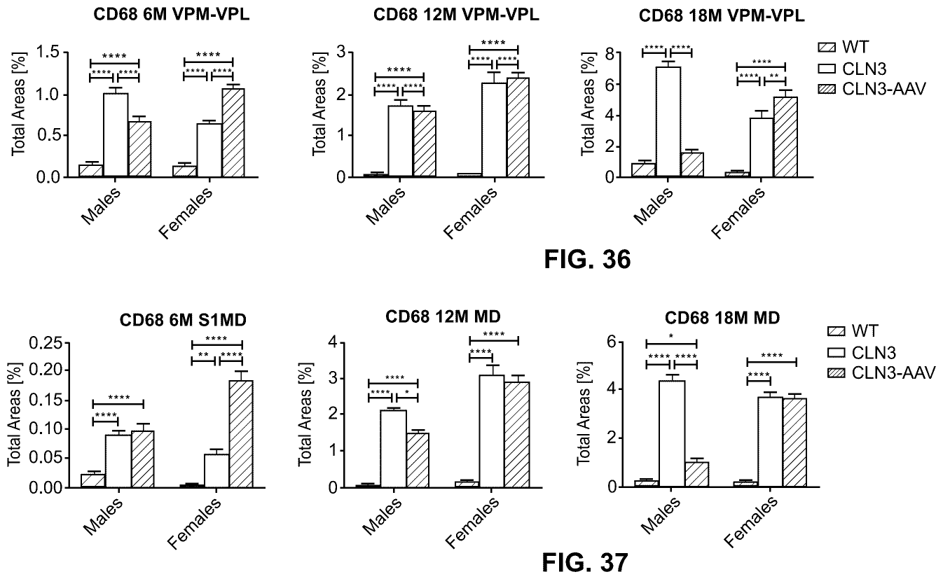

FIG. 37 demonstrates scAAV9.p546.CLN3 mice show differing levels of activated microglia (CD68⁺) in the Mediodorsal Nucleus (MD) based on sex. At 6 months of age, treated females had increased microglial activation compared to wild type and untreated females, with treated males higher than wild type but not different than untreated males. At both 12 and 18 months of age, treated males had higher microglial activation than wild type and lower activation than untreated males, while treatment appeared to have no effect on females.

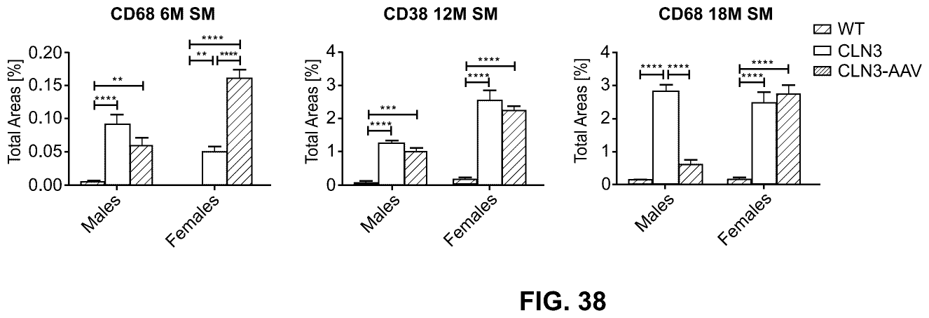

FIG. 38 demonstrates mice treated with scAAV9.p546.CLN3 showed differing levels of activated microglia in the Submedial Nucleus (SM) based on sex. At 6 months of age, treated females had increased microglial activation compared to wild type and untreated females, while treated males were not significantly different from untreated males, both having more activation than wild type. By 12 months, treatment appeared to not have an impact on degree of microglial activation in either sex. By 18 months, treated males have microglial activation reduced to wild type levels, while treated females remained activated to the same extent as untreated females.

The foregoing data demonstrates that scAAV9.p546.CLN3 treated animals have differential sex-based pathology related to ATP-Synthase Subunit C accumulation and CD68⁺ microglial activation in several regions of the Cln3^{Δ7/8} mouse brain. Differential sex-based pathological differences appear to be more female specific. The 12 month time point is where a majority of differences are most consistently seen, with many appearing only at 12 months and no longer present by 18 months.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

All documents referred to in this application are hereby incorporated by reference in their entirety.

1-71. (canceled)

72. A DNA plasmid comprising a recombinant adeno-associate virus (rAAV) genome, wherein the rAAV genome comprises a nucleotide sequence having at 90% sequence similarity to the nucleic acid sequence of SEQ ID NO: 4

73. The DNA plasmid of claim 72, wherein the DNA plasmid lacks AAV rep and cap genes.

74. The DNA plasmid of claim 72, further comprising a selectable marker.

75. The DNA plasmid of claim 72, wherein the rAAV genome is a self-complementary genome (scAAV).

76. The DNA plasmid of clam 72, wherein the rAAV genome is a single-stranded genome.

77. A cell comprising the DNA plasmid of claim 72.

78. The cell of claim 77, wherein the cell is a Hela cell, 293 cell, PerC.6 cell, MRC-5 cell, WI-38 cell, Vero cell, or FRhL-2 cell.

79. A method of making a recombinant adeno-associated virus (rAAV) for the treatment of CLN3-Batten Disease, comprising:
transferring the DNA plasmid of claim 72 into a cell;
producing rAAV particles encapsidating the DNA plasmid in the cell; and
isolating the rAAV particles from the cell.

80. The method of claim 79, wherein the cell is a Hela cell, 293 cell, PerC.6 cell, MRC-5 cell, WI-38 cell, Vero cell, or FRhL-2 cell.

81. The method of claim 79, further comprising a step of transferring AAV helper and AAV rep/cap plasmids into the cell.

82. The method of claim 81, wherein the rep/cap plasmid encodes for AAV9 capsid proteins.

83. An rAAV particle produced using the cell of claim 77.

84. An rAAV particle produced using the method of claim 79.


SEQUENCE LISTING (12 sequences)

SEQ ID NO: 1 - protein, 438 aa, Homo sapiens
  REGION 1..438: note = MISC_FEATURE - CLN3
MGGCAGSRRRFSDSEGEETVPEPRLPLLDHQGAHWKNAVGFWLLGLCNNFSYVVMLSAAHDILSHKRTSGNQSHVDPGPTPIPHNSSSRFDCNSVSTAAVLLADILPTLVIKLLAPLGLHLLPYSPRVLVSGICAAGSFVLVAFSHSVGTSLCGVVFASISSGLGEVTFLSLTAFYPRAVISWWSSGTGGAGLLGALSYLGLTQAGLSPQQTLLSMLGIPALLLASYFLLLTSPEAQDPGGEEEAESAARQPLIRTEAPESKPGSSSSLSLRERWTVFKGLLWYIVPLVVVYFAEYFINQGLFELLFFWNTSLSHAQQYRWYQMLYQAGVFASRSSLRCCRIRFTWALALLQCLNLVFLLADVWFGFLPSIYLVFLIILYEGLLGGAAYVNTFHNIALETSDEHREFAMAATCISDTLGISLSGLLALPLHDFLCQLS

SEQ ID NO: 2 - DNA, 1317 bp, Homo sapiens
  misc_feature 1..1317: note = CNL3
  source 1..1317: mol_type = genomic DNA
atgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctga

SEQ ID NO: 3 - DNA, 546 bp, synthetic construct
  misc_feature 1..546: note = Synthetic Polynucleotide
  misc_feature 1..546: note = P546 promoter
  source 1..546: mol_type = other DNA
gaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 4 - DNA, 2479 bp, synthetic construct
  misc_feature 1..2479: note = Synthetic Polynucleotide
  misc_feature 1..2479: note = CLN3 gene cassette
  source 1..2479: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaagtgttactaccggtatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 5 - DNA, 6008 bp, synthetic construct
  misc_feature 1..6008: note = Synthetic Polynucleotide
  misc_feature 1..6008: note = pAAV.P546.CLN3.KAN plasmid
  source 1..6008: mol_type = other DNA
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaagtgttactaccggtatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 6 - DNA, 106 bp, synthetic construct
  misc_feature 1..106: note = Synthetic Polynucleotide
  misc_feature 1..106: note = 5' ITR
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 7 - DNA, 100 bp, synthetic construct
  misc_feature 1..100: note = Synthetic Polynucleotide
  misc_feature 1..100: note = SV40 Intron
  source 1..100: mol_type = other DNA
ggtaagtttagtcttttttgtcttttatttcaggtcccggatccggtggtggtgcaaatcaaagaactgctcctcagtcgatgttgcctttacttctagg

SEQ ID NO: 8 - DNA, 147 bp, synthetic construct
  misc_feature 1..147: note = Synthetic Polynucleotide
  misc_feature 1..147: note = BGH Poly A
  source 1..147: mol_type = other DNA
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 9 - DNA, 141 bp, synthetic construct
  misc_feature 1..141: note = Synthetic Polynucleotide
  misc_feature 1..141: note = 3' ITR
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 10 - protein, 736 aa, synthetic construct
  REGION 1..736: note = Synthetic Polypeptide
  REGION 1..736: note = MISC_FEATURE - scAAV9 seuqence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - DNA, 23 bp, synthetic construct
  misc_feature 1..23: note = Synthetic Polynucleotide
  source 1..23: mol_type = other DNA
cgctagcatctcatcaggccttg

SEQ ID NO: 12 - DNA, 20 bp, synthetic construct
  misc_feature 1..20: note = Synthetic Polynucleotide
  source 1..20: mol_type = other DNA
agcatggacagcagggtctg